(19) United States
(12) Patent Application Publication
(10) Pub. No.: US 2023/0158196 A1
(43) Pub. Date: May 25, 2023

(54) Portable Clean Air Processor

(71) Applicants: Steven David Taylor, Harrington, WA (US); Steven Michael Cohen, New York, NY (US)

(72) Inventors: Steven David Taylor, Harrington, WA (US); Steven Michael Cohen, New York, NY (US)

(21) Appl. No.: 17/911,032

(22) PCT Filed: Mar. 11, 2021
(86) PCT No.: PCT/US2021/021975

§ 371 Date: Sep. 12, 2022

Related U.S. Application Data
(60) Provisional application No. 62/988,326, filed on Mar. 11, 2020.
(60) Provisional application No. 62/992,992, filed on Mar. 22, 2020.

(51) Int. Cl. A61L 9/16; A61L 9/014; A62B 7/10; A62B 9/00

(52) U.S. Cl. CPC A61L 9/16; A61L 9/014; A62B 7/10; A62B 9/003; A61L 2209/15; A61L 2209/111; A61L 2209/14; A61L 2209/22

(57) ABSTRACT

A portable air processing system that takes air from the ambient or other component, heats the air to a sufficient temperature so as to deactivate at least one type of pathogen or allergen, then cools the air to a comfortable breathing temperature, and then feeds this processed air under positive pressure to a controlled environment or mask covering the nose and mouth of a wearer, thus enabling one to breath cleaner air from a controlled source while mobile or stationary.

### FIELD OF INVENTION

The invention is in the field of public safety, more specifically in air processing for breathing.

### BACKGROUND OF INVENTION

There are many air processing systems and filter masks on the market.

Now, with the threat of airborne pathogens and exposure to infection in large gatherings of people outdoors and two or more people in enclosed spaces, there is a need for improved portable air processing systems that can help prevent the spread of infection, protect against particulates, or protect against other ailments.

### NOTES

PCT International application number: PCT/US2021/021975 filed on Mar. 11, 2021 Attorney Docket Number: NA

### BRIEF SUMMARY OF INVENTION

In a basic embodiment, the invention is an air processing system that enables air from the ambient or other device or system to enter a tube within which the air is cleaned, heated and cooled, processed, or filtered in some manner. In an air processing deactivating system, a heater heats the air to temperatures that can deactivate air-borne particles including pathogens and allergens. The air processing deactivating system has at least one heater section to raise the air temperature to achieve a predetermined temperature set point. The heating section is followed by a cooling section that cools the heated air back to a temperature that the user(s) can breathe the processed air in which pathogens and allergens have been deactivated. Within an air processing system, either an air processing deactivating system or an air processing filtering system is a pressure increasing fan or blower to provide positive pressure to the air supply tube that feeds a facemask or other volume in a portable system or to provide consistent airflow through an air processing system in a stationary room, building, or desktop version. In a portable air processing system, the wearer breathes air supplied to a facemask. Stationary embodiments are envisioned that can process air for workstations and or in larger facilities where an air processing system is stand alone or incorporated within an HVAC system for use in many spaces including where people meet, work, or congregate, or any enclosed space where a source of pathogen or allergen free air is necessary.

### BRIEF DESCRIPTION OF DRAWINGS

FIG. 1 shows a belt-attachable embodiment of an air processing system.

FIG. 2 shows a temperature profile of air as it passes though one portion of an air processing deactivating system.

FIG. 3 depicts a schematic representation of some components of an air processing deactivating system.

FIG. 4A shows a UV sterilizer employing a gas discharge tube that could be incorporated within some embodiments of an air processing deactivating system.

FIG. 4B shows a UV sterilizer employing LEDs or lasers that could be incorporated within some embodiments of an air processing deactivating system.

FIG. 5A shows two views of one embodiment of a heater oriented horizontally across the diameter of an air flow tube of an air processing deactivating system.

FIG. 5B shows two views of one embodiment of a heater oriented axially down the center of an air flow tube of an air processing deactivating system.

FIG. 5C shows two views of one embodiment of a serpentine heater oriented along a plane perpendicular to the flow of air in an air flow tube of an air processing deactivating system.

FIG. 5D shows two views of one embodiment of two heaters depicted in FIG. 5C that are at right angles to one another and each oriented perpendicularly to the flow of air in an air flow tubeof an air processing deactivating system.

FIG. 6A depicts the airflow disruptive effects of the heater configuration depicted in FIG. 5A of an air processing deactivating system.

FIG. 6B depicts a passive flow disrupter located before (upstream) the heater configuration depicted in FIG. 5B of an air processing deactivating system.

FIG. 6C depicts the heater configuration of FIG. 5A with an insulating wall surrounding the perimeter or external wall of the heater section pipe of an air processing deactivating system.

FIG. 7 depicts a double heater embodiment of the heater portion of the invention of an air processing deactivating system.

FIG. 8 depicts a reservoir bag or accumulator located in series with the of an air processing system.

FIG. 9 depicts a filter in series with the air processing system.

FIG. 10 shows a schematic representation of a quick disconnect air flow tube with mating elements of an air processing system.

FIG. 11 depicts a one-way valve in some embodiments of the facemask of an air processing system.

FIG. 12 depicts a larger more general embodiment of the invention with many features that could be incorporated in different embodiments of an air processing deactivating system ranging from expensive systems to cheaper wearable systems for the general public.

FIG. 13 depicts a double heater feeding a reservoir containing a serpentine piping arrangement with intermediate heaters to maintain at least a preset temperature within the reservoir within an air processing deactivating system.

FIG. 14 depicts a heat exchanger allowing heat to transfer from a hot air stream to a cold air stream, thus lowering the temperature of the hot air stream and raising the temperature of the cold air stream within an air processing deactivating system.

FIG. 15 depicts another general embodiment of an air processing deactivating system incorporating a heat exchanger and a heated insulated reservoir.

FIG. 16A shows a temperature profile of air as it passes though an embodiment of an air processing deactivating system depicted in FIG. 15.

FIG. 16B shows an example of a temperature profile of the hot and cold air streams passing through the heat exchanger of an air processing deactivating system.

FIG. 17 depicts one configuration of a reservoir composed of slats within a rectangular box, thus creating rectangular tubes through which the air passes during its residence time within the reservoir of an air processing deactivating system.

FIG. 18 depicts the cross-section of one embodiment of a heat exchanger of an air processing deactivating system consisting of a tube within a tube.

FIG. 19 depicts one embodiment of a non-portable air processing system that can be mounted to a workstation wherein air can be drawn into the air processor from one location and exit through an output hose to an exit nozzle directing a flow of processed air into the face of an operator.

FIG. 20 depicts one embodiment of a self-contained desktop air processing system.

FIG. 21 depicts one embodiment of a desktop air processing system with a remotely-positionable exit nozzle being fed via a connecting hose.

FIG. 22 depicts an output connecting hose as depicted in FIGS. 19 and 21 entering a distribution manifold with multiple output nozzles or zones containing air-direction louvres.

FIG. 23 depicts a left output nozzle with directable air-direction louvres and a right output nozzle with directable air-direction louvres.

FIGS. 24A and 24B depict some elements in a large installation for processing and distributing large quantities of air to many people such as those in a plane, train, bus, or other controlled or random seating or standing arrangement from an air processing deactivating system or an air processing filtering system.

FIG. 25 depicts an airflow philosophy in a large space wherein air is processed in an air processor, exits as an output airstream which generally blows in one direction, in this case from above, people then breath the processed air, and exhaled air then flows into an exhaust airstream that either exhausts all or a portion of the air to the ambient or draws all the air or a portion thereof back into the air processing system for reprocessing.

### SUMMARY OF INVENTION

In a basic embodiment the invention is an air processing system that enables air from the ambient to enter a tube within which a heater heats the air to temperatures that can kill pathogens. This involves at least one heater section with temperature sensors to monitor the input temperature as well as intermediate system temperatures so that the proper amount of power can be dispensed to raise the air temperature as high as is necessary to achieve a temperature set point. This is followed by a cooling section that cools the heated air back to the starting temperature or slightly above the starting temperature so the wearer in portable systems can breathe processed air in which pathogens have been killed. At the output of the cooling section is a pressure increasing output fan or blower to provide positive pressure to the air supply tube that feeds a facemask in which positive pressure is being maintained. In a portable unit or workstation the wearer breathes air supplied to the facemask. Stationary embodiments are envisioned that can process air for workstations and or in larger facilities where the system is incorporated within an HVAC system for use in airplanes, buses, trains, cars, ships, hospitals, schools, public buildings, factories, assembly lines, meat packing plants, food processing plants, any building where people congregate, houses, or any enclosed space where a source of pathogen free air is necessary.

Not all viruses and pathogens respond the same way to heat and temperature, and different temperatures kill different viruses and pathogens. For instance, in a scientific paper entitled Inactivation of the novel avian influenza A (H7N5) virus under physical conditions or chemical treatment, authored by Shumei Zou, Junfeng Guo, Rongbao Gao, Libo Dong, Jianfang Zhou, Ye Zhang, Jie Dong, Hong Bo, Kun Qin, and Yuelong Shu, the following temperatures and corresponding times of virus inactivation were measured:

| Temperature | number of | number of |
|---|---|---|
| In degrees | minutes to | seconds to |
| Centigrade | deactivation | deactivation |
| 56 | 30 | 1800 |
| 65 | 10 | 600 |
| 70 | 1 | 60 |
| 75 | 1 | 60 *(this is an |
| | | overkill condition) |
| 100 | 1 | 60 *(this is an |
| | | overkill condition) |

The tests failed to resolve time less than one minute, but one can extrapolate the above temperatures and corresponding times in seconds. For the purpose of simplifying the graph,

56 degrees C. is taken as 0 as a reference datum,

65 degrees C. is a ΔT of 9 degrees C. above 56 degrees C., and

70 degrees C. is a ΔT of 14 degrees C. above 56 degrees C.

The results are graphed below:

Using the above equation, a Δ T of 14.603 degrees above the datum temperature, or 70.603 degrees C. or 159.08 degrees F. should kill H7N5 in 1 second, and a ΔT of 14.614 above the datum should kill the virus immediately. This corresponds to 70.614 degrees C. or 159.1 degrees F. Initial data indicates that the Covid-19 is more fragile than H5N7. If temperatures above 85 degrees C. or even 100 degrees C. are generated, this can be an effective portable device for producing safer air to breathe.

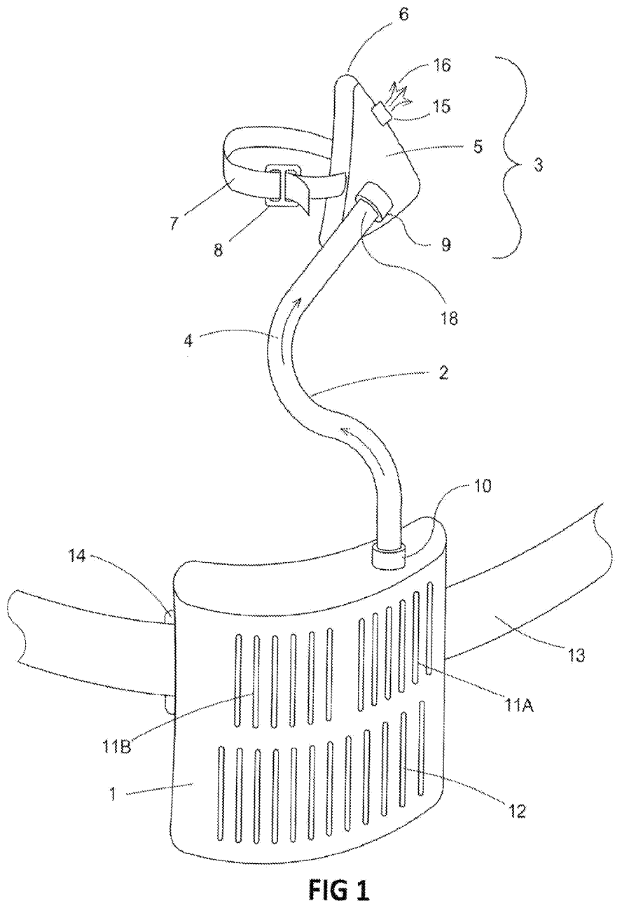

FIG. 1 shows a belt-attachable embodiment of an air processing system. Shown is the air processing deactivation system with an internal battery 1. Air enters the air processing deactivation system with an internal battery 1 at the input air vent 12 and exits after having passed through a series of tubes in which the air was heated and cooled, which then passes through the connecting tube or connecting hose 2 to a positive pressure facemask with attachment elastic adjustable straps 3 which is flooded with positive pressure air 4 so that all air breathed by the wearer comes through the air processing deactivation system with an internal battery 1 and little to no air comes from the ambient which can be infected. The positive pressure facemask with attachment elastic adjustable straps 3 comprises a shell 5, a cushion 6, at least one one-way valve 15, a hose coupler at facemask 9, at least one strap 7, and at least one adjustment buckle 8 can be used for both the air processing deactivation system and the air processing filtration system. The cushion 6 may be made from a low durometer rubber-like material and is at least one of in whole or in part silicone rubber, latex, urethane, foam, or other material with a durometer permitting a comfortable interface between the shell 5 and the face of the wearer. The goal is not to have a perfect seal allowing no airflow as in a scuba mask, but to contain the positive pressure air 4 so that all inhaled air has passed through the air processing system with an internal battery 1. In some embodiments at least one strap 7 is multiple straps and adjustable via at least one adjustment buckle 8 to secure the positive pressure facemask with attachment elastic adjustable straps 3 to the face and head of the wearer in a stable and comfortable manner. In some embodiments, a backflow one-way valve 18 (not shown) will be incorporated to permit airflow in one direction towards the positive pressure facemask with attachment elastic adjustable straps 3 but to prevent exhaled air, saliva, vomit, blood, sebaceous secretions, mucous, water, liquids, solids, suspensions, other material, or any combination thereof from flowing back into an air processing system with an internal battery 1. This backflow one-way valve 18 can be located in at least one of: the connecting hose 2, the hose coupler at the facemask 9, the output orifice 17 (not shown) supplying positive pressure air 4 to the positive pressure facemask with attachment elastic adjustable straps 3, the hose coupler at of air processing system with an internal battery 10. Also shown is a cooling input vent 11A or slats that enables ambient air to enter the air processing deactivating system with an internal battery 1 to be used as cooling air, but at no time does ambient air mix with processed and isolated air to be breathed. And also shown is a cooling exhaust vent 11B of an air processing deactivating system.

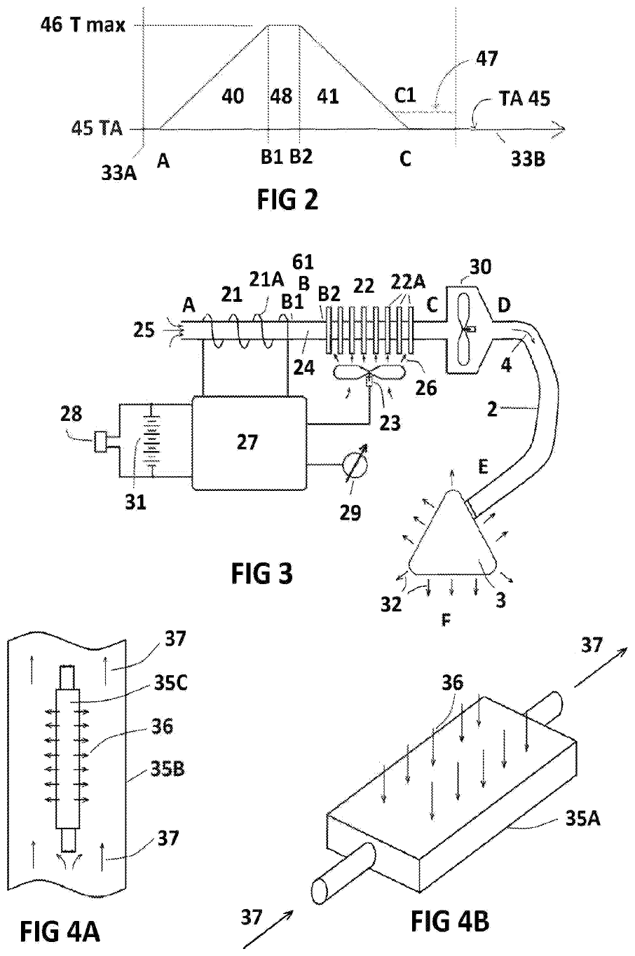

FIG. 2 shows the temperature cycle or profile in one embodiment of an air processing deactivating system. Temperature 33A is on the vertical axis and length down the airflow pipe Lap 33B is on the horizontal axis.

FIG. 2 also shows the maximum temperature Tmax 46 which is the highest predetermined temperature achieved during and at the end the heating cycle 40. A small temperature sustaining heat reservoir 61 is depicted as region “B” which is further broken down into points “B1”, the output from air heater 21 and “B2”, the input to air cooler 22. The temperature sustaining heat reservoir 61 is maintained at reservoir temperature Tr 48, which will usually be the highest predetermined temperature Tmax 46.

FIG. 3 depicts a schematic representation of some components of one embodiment of the air processing deactivating system. Shown is the control circuit 27, which controls power to the representative heating element 21A in the air heater 21 and the air cooling fan 23. There will also be a mumber of sensors not shown that will be connected to the control circuit 27. Input air 25 is drawn from into the airflow pipe 24, and in many cases this will simply be the ambient input air 19 after first passing through the input air vent 12 (shown in FIG. 1). Input air may pass through filter 59 (shown in FIGS. 9, 12, and 15). Input air 25 enters the airflow pipe 24 at point “A”. Input air 25 passes through the air heater 21 portion of the invention where heat is added to raise the temperature of the air from the ambient temperature Ta 45 to the highest predetermined temperature Tmax 46 at point “B1. The heating cycle 40 of this heating process is shown in FIG. 2.

Many viruses are deactivated when elevated to temperatures at or above 20 to 65 degrees Centigrade (68-149 degrees Fahrenheit). If air is heated above this range to 50-100 degrees Centigrade above ambient temperature, this ensures that the air will have been heated well above the temperature necessary to kill many pathogens that can infect people. If the starting ambient temperature is 62 degrees Fahrenheit, a 100 degree Centigrade temperature rise would correspond to 242 degrees Fahrenheit. From point “B2” air passes through the air cooler 22, with the goal being to cool the air back to the ambient temperature Ta 45 at point “C” or even temperature higher than ambient Tc1 (point “C1” in FIG. 2) in some embodiments if the wearer desires breathing room temperature air when in environments colder than room temperature. These different cooling cycle 41 temperatures are also shown in FIG. 2 wherein the final cooling temperature can either be ambient temperature Ta 45 (point “C”) or temperature higher than ambient Tc1 47 (point “C1”). In one scenario air might enter the system at 20 degrees Fahrenheit at point “A”, be elevated to 232 degrees Fahrenheit at point “B”, then be cooled to 70 degrees for comfortable breathing air at point “C1” settable by the user or predetermined. In the embodiment of an air processing deactivating system depicted, air then passes through a pressure out fan 30 to increase the pressure between point “C” or “C1” and point “D” before entering the connecting hose 2 to enter the positive pressure facemask with attachment elastic adjustable straps 3 at point “E”. In this schematic representation the pressure out fan 30 is shown to be an axial fan for example only. In actual implementation this may require a centrifugal fan or blower to produce a higher static pressure rise. There will be a lot of air (fluid) pressure loss in this system so for positive pressure to be maintained at the positive pressure facemask with attachment elastic adjustable straps 3 the appropriate fan is chosen. In the positive pressure facemask with attachment elastic adjustable straps 3, positive pressure is being maintained to be breathed by the wearer, and all output air from facemask 32 comprises both leaked air and exhaled air which exits either around the perimeter of the mask or via one way valve 15. Some embodiments of the facemask can incorporate at least one one-way valve 15 as depicted in FIGS. 1 and 11. The control circuit 27 can turn the air heater 21 on and off and modulate the power in a PWM or other fashion. The cooling fan 23 can also be turned on and off or employ PWM control to adjust the cooling air 26 past at least one cooling fin 22A. Not shown in this drawing is at least one temperature sensor in at least one location so that thermal feedback exists and both heater power and the cooling function can be controlled on a breath by demand basis by the control circuit 27.

In FIG. 3, at least one filter 59 for additional cleansing of the air may be added at any location (for example at locations A, B, C, D, or E) that can be incorporated within some embodiments of an air processing deactivating system. This will allow the same air processing deactivating system to be used for multiple applications and situations and may add additional air cleaning for virus, pathogen, pollen, smoke, dust, and airborne suspended particulate removal. In FIG. 3, at least two filters 59 for cleansing of the air are added in parallel at any location (for example at locations A, B, C, D, or E) that are incorporated in all embodiments of an air processing filtering system. In addition, all heating and cooling sections and the associated fan 23 are removed in all embodiments of an air processing filtering system. This will allow the airflow to be maintained if one of the filters become blocked, clogged, or broken in any manner.

Other embodiments of an air processing system are envisioned in which an internal or external air cooler 22 further incorporates components that can lower the temperature below ambient temperature so ambient input air 19 at a starting ambient temperature Ta 45 can be lowered so cool comfortable air can be delivered to the mask 3 or environment 32.

In addition, if multiple up and down temperature cycles are needed to ensure removal of a virus or pathogen (or other material) in an air processing deactivating system, a return airflow path with on/off valves may be added between, before, or after any points within the air heater 21, air cooler 22, cooling fan 23, connecting hose 2, positive pressure facemask with attachment elastic adjustable straps 3 system (A, B, C, D, and E).

FIG. 4A shows a discharge tube UV sterilizer 35B employing a gas UV discharge tube 35C that could be incorporated within some embodiments of an air processing system. FIG. 4B shows a solid state UV sterilizer 35A employing LEDs or lasers that could be incorporated within some embodiments of the invention. UVC would be the preferable band, and embodiments could incorporate discharge tubes that can produce 256 nanometers which can act as a pre-sterilizer within a system that can be both portable and non-portable. FIG. 4A depicts an embodiment of of an air processing system employing a gas UV discharge tube 35C (florescent) centrally located within airflow pipe Lap 33B, which is a tube of circular cross-section. The system airflow air 37 flows between the inside diameter formed by the UV discharge tube 35C and the inside wall of the airflow pipe Lap 33B cylinder within which air flows. LED sterilizers can operate at small wavelengths and this can kill certain microbes and could easily be incorporated in some embodiments of the invention. Lasers can also be used in some embodiments and can produce short wavelengths at ever-increasing efficiencies.

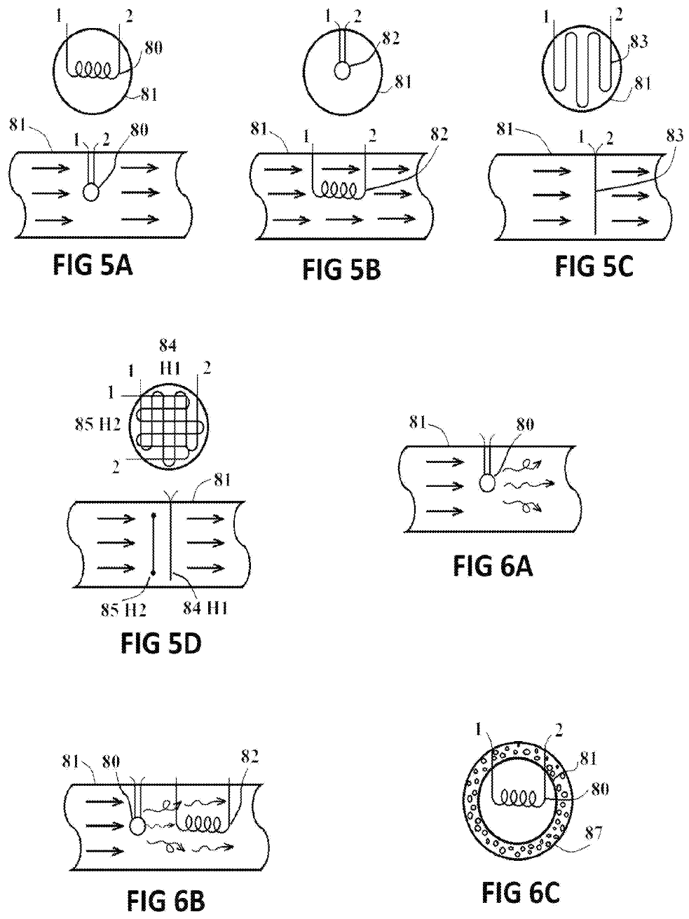

FIG. 5A shows two views of one embodiment of an air processing deactivating system with a diameter oriented heater coil 80 which is oriented horizontally across the diameter of an airflow pipe 81. The top view is looking down the airflow pipe 81 into the direction of airflow. The diameter oriented heater coil 80 can be seen in this view, with one end of the diameter oriented heater coil 80 depicted as point 1 and the other end of the diameter oriented heater coil 80 depicted as point 2. The lower drawing shows the cross-section of the airflow pipe 81 as viewed perpendicular to the direction of airflow, looking into the diameter oriented heater coil 80.

FIG. 5B shows two views of one embodiment air processing deactivating system with an axially oriented heater coil 82 oriented axially down the center of an airflow pipe 81. The top view is looking into the airflow pipe 81 into the direction of airflow. An axially oriented heater coil 82 can be seen in this view, looking into the axially oriented heater coil 82 which is oriented down the length of the airflow pipe 81. One end of the axially oriented heater coil 82 is depicted as point 1 and the other end of the axially oriented heater coil 82 is depicted as point 2. The lower drawing shows the cross-section of the airflow pipe 81 as viewed perpendicular to the direction of airflow, looking at the axially oriented heater coil 82 sideways.

FIG. 5C shows two views of one embodiment of an air processing deactivating system with a vertically oriented serpentine heater coil 83 oriented along a plane perpendicular to the flow of air in an airflow pipe 81. The top view is looking down the airflow pipe 81 into the direction of airflow. The vertically oriented serpentine heater coil 83 can be seen in this view, with one end of the vertically oriented heater coil 83 depicted as point 1 and the other end of the vertically oriented heater coil 83 depicted as point 2. The lower drawing shows the cross-section of the airflow pipe 81 as viewed perpendicular to the direction of airflow, looking into the vertically oriented heater coil 83 edgewise.

FIG. 5D shows two views of one embodiment of an air processing deactivating system including two serpentine heaters depicted in FIG. 5C that are at right angles to one another and each oriented perpendicularly to the flow of air in an airflow pipe 81. Heater coil H1 84 is vertically oriented and heater coil H2 85 is horizontally oriented. Both serpentine heater coils can be seen in this view, with one end of heater coil H1 84 depicted as point 1 and the other end of the heater coil H1 84 depicted as point 2, and likewise with heater coil H2 85. The lower drawing shows the cross-section of the airflow pipe 81 as viewed perpendicular to the direction of airflow, looking into both heater coils edgewise and offset from one another. This embodiment could enable heating to occur closer to the farthest moving streamlines of air.

FIG. 6A depicts the airflow disruptive effects of the heater configuration of an air processing deactivating system depicted in FIG. 5A. What is desired in a moving flow system such as this is to actually create turbulence rather than laminar flow so the heat transfer can be improved because the farthest streamline of air in laminar flow must be heated above the temperature of lethality to a pathogen within the air. This would mean that the streamlines closer to the heater would have to be heated way above the maximum temperature to guarantee that all the air in a laminar flow system were to exceed the minimum necessary temperature. Some embodiments of the heater portion of the system can be combinations of heater orientations such as those depicted in FIGS. 5A and 5B, as well as 5C and 5D. In addition, flow disrupters can be placed within the airflow to cause turbulence. This can take the form of protrusions and depressions in the airflow pipe 81. Wads of porous material or steel wool can be placed within the airflow pipe 81 to cause irregular airflow patterns.

FIG. 6B depicts a passive flow disrupter located before (upstream) the heater configuration depicted in FIG. 5B of an air processing deactivating system. It should be noted that there can be any number of flow disrupters and heater orientations and the examples shown are for illustrative purposes only. The goal is to mix the air so all parts of the air can be raised in temperature and too extreme a temperature profile can be avoided.

FIG. 6C depicts the heater configuration of an air processing deactivating system of FIG. 5A shown for example with an insulating wall 87 surrounding the perimeter or external wall of the heater section airflow pipe 81. The more heat loss prevented the more heat will be delivered to the air flowing within the airflow pipe 81. After some time the temperature of the pipe wall will reach or approach a steady state operating temperature as a function of axial length down the airflow pipe 81. The airflow pipe 81 itself can be made of a ceramic material or have ceramic sections to enable higher temperature operation.

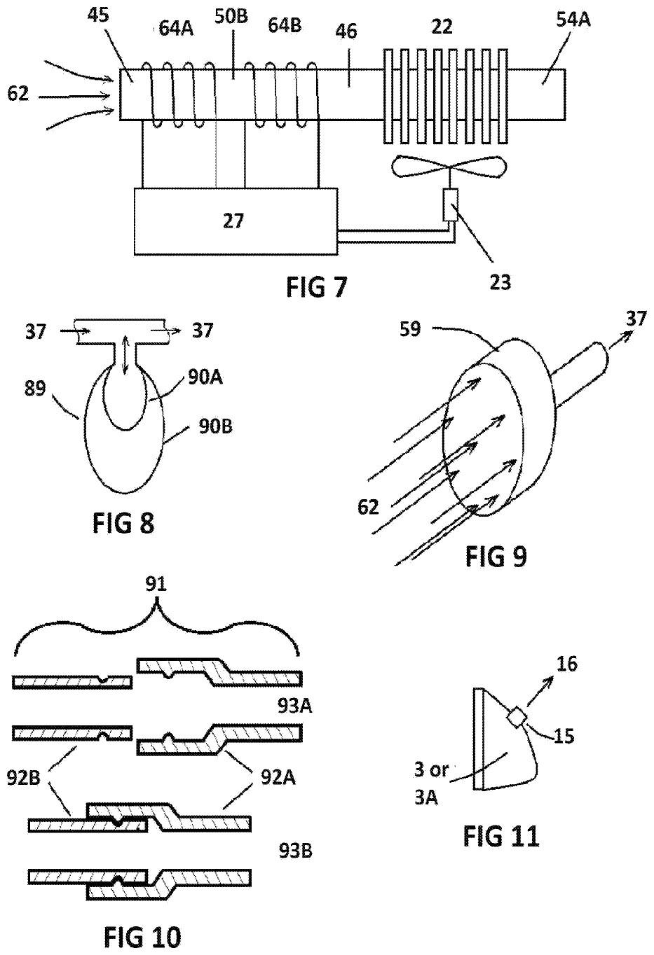

FIG. 7 depicts a double heater embodiment of the air heater 21 portion of an air processing deactivating system. In this embodiment air in 62 of ambient temperature Ta 45 enters heater H1 64A and exits at heater 2 input temperature 50B. By knowing the temperature difference between heater 2 input temperature 50B and ambient temperature Ta 45 and knowing how much energy was used in heater H1 64A, the amount of energy necessary to be expended in heater H2 64B can be calculated in real time and known. If E1 energy was expended in heater H1 64A to produce a 75 degree temperature rise, then knowing the target maximum temperature Tmax 46 (point “B” in FIG. 3) and more specifically maximum temperature Tmax 46—heater 2 input temperature 50B, then E1/3 is the supplemental energy to be expended if it is desired to raise the air temperature an extra 25 degrees. Energy of course is power x time, so energy directly maps into instantaneous power, and energy expended during a breath of air is the integrated power throughout the breath. This can occur in real time, and learning the breathing habits and pulse and surge demands can be used to predict the profile of energy expenditure so the target temperature or a higher temperature can be reached. In a system such as this as long as the cooling capacity temperature cooler out Tco 54A—maximum temperature Tmax 46 exceeds the highest predetermined maximum temperature Tmax 46 temperature that can be reached, it doesn't matter if some energy is wasted as long as there is battery capacity to spare given the time the system must perform between charges. Also, in some embodiments of an air processing system with a cooling fan that can be pulsed, turned on and off as needed, as well as employ PWM control.

FIG. 8 depicts a reservoir bag or accumulator 89 located in series with the system airflow air 37 of an air processing system. All air processing systems (air processing deactivating system or an of an air processing filtering system) can incorporate an accumulator 89 to act as an expanding and contracting reservoir of processed air before or after the output orifice. Shown is the uninflated state 90A and the inflated state 90B. This can smooth out the pulsed breathing action of the way people actually breath. The accumulator can either be internal to the controller unit or external, though this is fully sterilized and processed air so the valving must be such that there is no leakage to allow ambient air to accidentally enter the system. A stopcock or shutoff valve (not shown) will be employed if the bag is removed from the system airflow air 37 in such embodiments.

FIG. 9 depicts a filter 59 in which air from air in 62 is drawn into the filter 59, which passes filtered air out to feed the system airflow air 37. This can be a pancake style filter with a large area filter necking down to the general system plumbing diameter. The filter contains a mechanical porous filter that is the equivalent of an N95 or N99 filter, Some embodiments of either air processing systems will incorporate a MERV 16 or better material. Some embodiments will contain a granulated carbon filter as well. The entire filter 59 or components of the filter 59 are replaceable. The filter 59 blocks droplets of moisture as well as particulate material, dust, sand, soot, fly ash, and smoke.

FIG. 10 shows a schematic representation of the cross-section of an airflow tube quick disconnect 91 with male mating element 92A and female mating element 92B of either air processing system. Shown is the disconnected position 93A and the connected position 93B. There are many pop-on/pop-off air connectors that can be employed for easy connection and disconnection and replacement of system components including but not limited to the filter 59, accumulator 89, connecting hose 2 at either end, and the positive pressure facemask 3A. Some embodiments of an air processing system have a bayonet style connector in which a male end is inserted into a female receiver, and rotated to click into a locked position.

FIG. 11 depicts a one-way valve 15 in some embodiments of an air processing system of a positive pressure facemask with attachment elastic adjustable straps 3 or positive pressure facemask 3A. There can be more than one one-way valve 15 employed to permit exhaled air and positive pressure air 16 to be discharged into the ambient and prevent air from the ambient from entering the positive pressure facemask 3A during inhalation. In general, it is the goal to provide positive pressure in sufficient quantity so that an inhaled breath can be taken without air starvation within the positive pressure facemask 3A at peak flow demand. Other facemasks incorporate both a one-way valve 15 and a facemask filter 94 (not shown) and it is understood that the use of the term “one-way valve” will be a one way-valve with a facemask filter 94 or only a facemask filter 94 to prevent or minimize expulsion of droplets emanating from the wearer's mouth and nose.

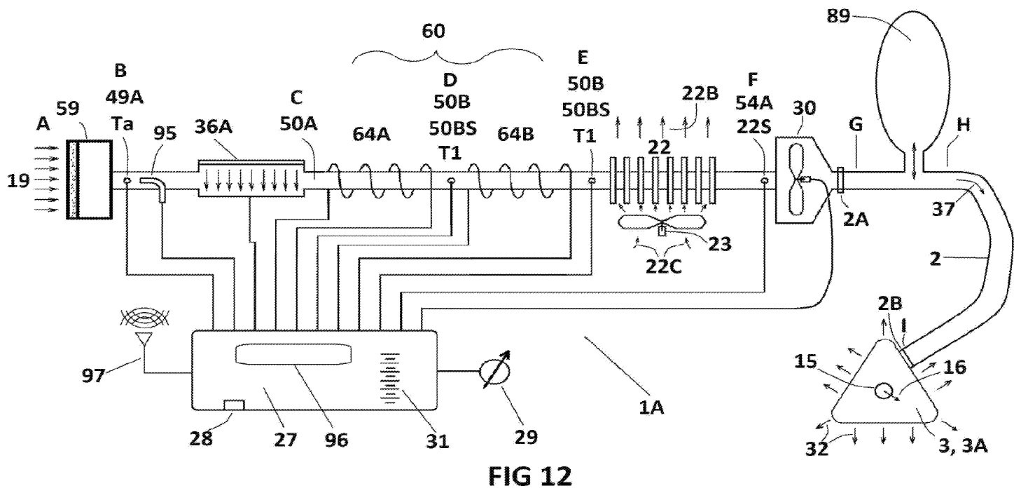

FIG. 12 depicts a larger more general embodiment of an air processing deactivating system with many features that could be incorporated in different combinations and embodiments ranging from expensive air processing deactivation systems 1A to cheaper wearable air processing deactivating systems 1A for the general public. Shown is ambient air input 19 at point “A” entering the filter 59 as described in FIG. 9. Ambient temperature Ta sensor 45A located at point “B” measures the ambient input temperature Ta 45 at the output of filter 59 and sends this value to the control circuit 27 where it is transformed into at least one digital and/or analog level to be used to control and monitor the system, as are all temperature sensors. System airflow air 37 speed is measured by an airflow sensor 95 and fed to the control circuit 27. The system airflow air 37 then enters the UV illuminator 36A as described in FIGS. 4A and 4B, which is turned on and off or modulated by the control circuit 27. UV output 50A at point “C” is the output air of the UV illuminator 36A and input to the heater section 60, the first section of which is heater H1 64A, which is turned on and off or modulated by the control circuit 27. The temperature output of heater H1 64A is sensed by heater 2 input temperature sensor SOBS at point “D”, which is fed to the control circuit 27. The system airflow air 37 then enters the second section of heater section 60 which is heater H2 64B, which is turned on and off or modulated by the control circuit 27, and exits at maximum temperature Tmax 46 which is measured by heater H2 output temperature sensor 64BS at point “E”, which is fed to the control circuit 27. The system airflow air 37 then enters the air cooler 22, and the cooling fan 23 is controlled by the control circuit 27. The cooling fan 23 draws ambient input cooling air 22C from the cooling input vent 11A as shown in FIG. 1 and expels warm cooling air output 22B from the cooling exhaust vent 11B as shown in FIG. 1. Air cooler output temperature sensor 22S located at point “F” is fed to the control circuit 27 and measures the output temperature of the air cooler 22, which is the input point “F” to the pressure out fan 30 which is turned on and off or modulated by the control circuit 27. The output of the pressure out fan 30 is point “G”. The connecting hose 2 attaches to a point after the output of the pressure fan out 30 via connecting hose bayonet connector first end 2A. The accumulator 89 is in series with the connecting hose 2 located between points “G” and “F” within the system airflow air 37. It is shown at this location for example only and can be located anywhere after the pressure out fan 30 including at the positive pressure facemask 3A itself. The accumulator 89 is shown for a more general embodiment of the invention but other embodiments of the invention will have no accumulator 89. The system airflow air 37 then continues on to connecting hose bayonet connector second end 2B at point “I” and the input to the positive pressure facemask 3A where positive pressure is being maintained. The positive pressure facemask 3A in this embodiment incorporates at least one one-way valve 15 as depicted in FIG. 11. The positive pressure facemask 3A or positive pressure facemask with attachment elastic adjustable straps 3 shows the at least one one-way valve 15 which permits exhaled air and positive pressure air 16 to escape as well as all output air from facemask 32 which can escape from all exits including the cushion 6.

The control circuit 27 incorporates a temperature control 29, and in this embodiment of an air processing deactivating system that also has a display 96 readout of pertinent data. The box which houses the control circuit 27 contains a rechargeable battery 31 which can be a removeable rechargeable battery 31. In some embodiments the rechargeable battery 31 will be more than one battery so that one rechargeable battery 31 or battery pack 31A can be removed and swapped with another rechargeable battery 31 or battery pack 31A while the remaining rechargeable battery 31 or battery pack 31A powers the air processing system 1A. Also shown is a charging jack 28. In this embodiment there is also a WIFI communication 97 subsystem that can enable pertinent data and logged information to be sent in one direction or bidirectionally to at least one other Bluetooth operated device such as a Bluetooth fed audio speaker or a smart device such as a phone, tablet, or computer.

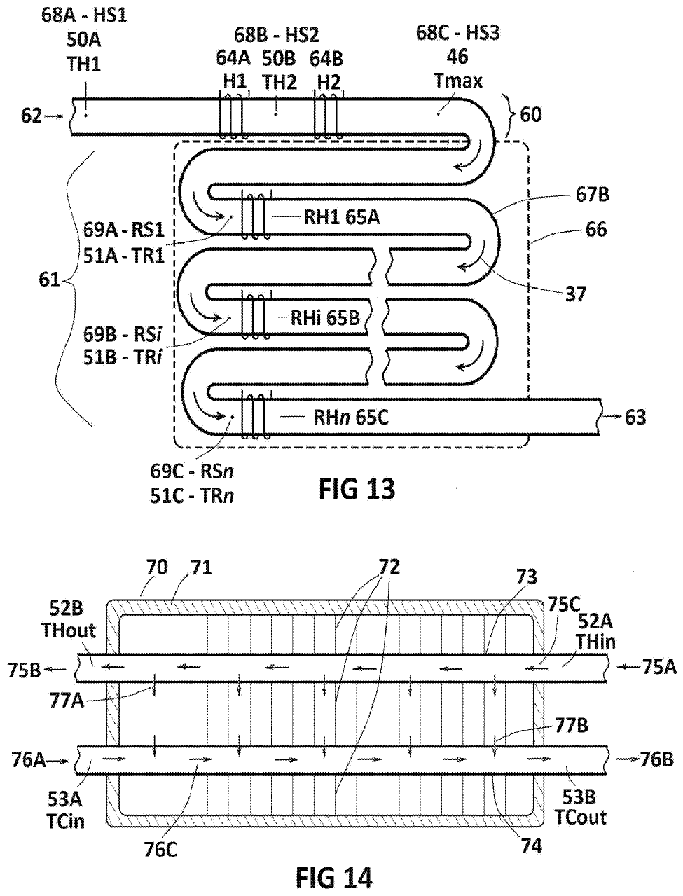

FIG. 13 depicts a double-heater heater section 60 of an air processing deactivating system heating a flow of air in 62 which enters a temperature sustaining heat reservoir 61 containing a serpentine piping arrangement with intermediate heaters to maintain at least one maximum predetermined temperature Tmax 46 within the temperature sustaining heat reservoir 61.

This embodiment shows a temperature sustaining heat reservoir 61 with supplemental heaters, but the temperature sustaining heat reservoir 61 could be non-heated if the heat reservoir insulation 66 prevents heat loss from the temperature sustaining heat reservoir 61 such that the maximum temperature Tmax 46 will remain substantially the same during air residence time within the temperature sustaining heat reservoir 61. The residence time in the temperature sustaining heat reservoir 61 depends on both the reservoir air pipe 67B cross-sectional area, the length of reservoir air pipe 67B, and the demand for air in volume per unit time. The faster air passes through the temperature-sustaining heat reservoir 61, the shorter the residence time within the temperature sustaining heat reservoir 61. Air will flow faster in a small diameter reservoir air pipe 67B than a large diameter air reservoir pipe 67B, and doubling the length of an air reservoir pipe 67B doubles the residence time that air spends in the temperature sustaining heat reservoir 61.

Depicted in this embodiment of an air processing deactivating system is air in 62 entering the heater air pipe 67A from the left at heater 1 input temperature TH1 50A as measured by heater temperature sensor HS1 68A. This can be air in 62 from any number of sources including but not limited to air in 62 from the ambient, air from a filter 59, air from a UV sterilizer illuminating system airflow air 37 with UV light 36, air from a heat exchanger 70, air from another heater, or air from any other source. Heater H1 64A and heater H2 64B are schematically represented heaters in the heater section 60 and produce heat which raises the temperature of the air in 62 as the air in 62 passes through the heater air pipe 67A. Heat from heater H1 64A is absorbed by air in 62 and the temperature is raised to heater 2 input temperature TH2 50B as measured by heater temperature sensor HS2 68B. Heat from heater H2 64B is absorbed by air in 62 and the temperature is raised to maximum temperature Tmax 46 as measured by heater temperature sensor HS3 68C.

The maximum predetermined temperature Tmax 46 is the temperature at the output of heater H2 64B and prior to entry to the input of the temperature sustaining heat reservoir 61. The temperature sustaining heat reservoir 61 contains the reservoir air pipe 67B, which can be any cross-section, diameter pipe or channel and can be any length such that it produces the residence time necessary to deactivate pathogens.

Shown are three representative left and right lengths of reservoir air pipe 67B. Shown is a first left and right pair of reservoir air pipe 67B with reservoir temperature sensor RS1 69A indicating reservoir temperature TR1 51A. If there is heat loss from the reservoir air pipe 67B as determined by a temperature difference between reservoir temperature TR1 51A and maximum temperature Tmax 46, this will trigger an infusion of heat generated in heater H2 64B to raise the temperature of flowing air back to maximum temperature Tmax 46. There can be a second, third, or more set of reservoir pipes, so a representative ith left and right pair of pipes is shown with a reservoir temperature sensor RSi 69B indicating reservoir temperature TRi 51B. And there could be other reservoir pipes following this, and lastly is shown the nth left and right pair of pipes with a reservoir temperature sensor RSn 69C indicating reservoir temperature TRn 51C. The difference between maximum temperature Tmax 46 or reservoir temperature TRi 51B and reservoir temperature TR1 51A will trigger the infusion of heat generated in reservoir heater RH1 65A to raise the temperature of flowing air back to maximum temperature Tmax 46. The difference between maximum temperature Tmax 46 or reservoir temperature TRn 51C and reservoir temperature TRi 51B will trigger the infusion of heat generated in reservoir heater RHi 65B to raise the temperature of flowing air back to maximum temperature Tmax 46. And lastly, the difference between maximum temperature Tmax 46 or reservoir temperature TRout 51D and reservoir temperature TRn 51C will trigger the infusion of heat generated in reservoir heater RHn 65C to raise the temperature of flowing air back to maximum temperature Tmax 46.

In this scenario the respective heaters will be turned on should the temperature begin dropping due to heat loss anywhere within the reservoir air pipe 67B. During the start-up temperature transient many of the heaters will be turning on and off to compensate for the initial heat loss from the heater air pipe 67A, the reservoir air pipe 67B, and insulation such as the heat reservoir insulation 66 or other locations in the system.

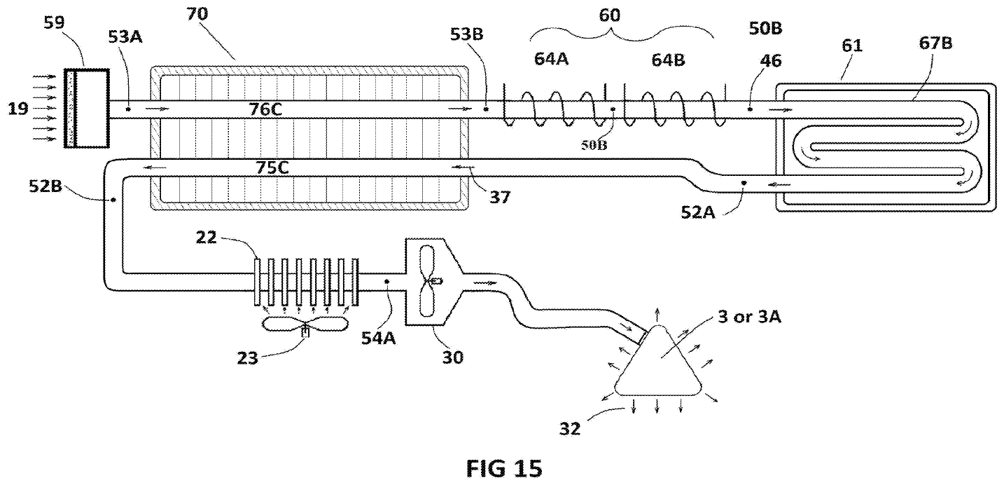

FIG. 14 depicts an insulated heat exchanger 70 of an air processing deactivating system allowing heat to transfer from a high temperature pipe 73 to a low temperature pipe 74, thus lowering the temperature of the hot air stream 75C and raising the temperature of the cold air stream 76C. This heat exchanger 70 is incorporated within the embodiment depicted in FIG. 15, which depicts another general embodiment of the invention incorporating a heat exchanger 70 and a temperature sustaining heat reservoir 61. As already explained in FIGS. 3, 7, and 12, at least one heater heats the system airflow air 37 to some maximum temperature Tmax 46, and then the system airflow air 37 is cooled back to ambient temperature Ta 45 or or a comfortable breathing temperature such as room temperature or a temperature higher than ambient Tc1 or in other embodies temperatures lower than ambient. The heat contained in the high temperature pipe 73 is used to preheat the system airflow air 37 entering the first heater H1 64A heating coil, thus reclaiming a portion of energy that would otherwise be lost forever in the cooling cycle 41. This extends the battery life as well. In a perfectly adiabatic system with perfect heat transfer the amount of heat entering the low temperature pipe 74 used to heat the system airflow air 37 would be exactly the amount of heat being given up by the system airflow air 37 in the high temperature pipe 73 to return the system airflow air 37 to the same starting ambient temperature Ta 45 of the ambient input air 19. In the real world, a heat exchanger will reclaim only a portion of the energy required to heat the air, but any reclamation will extend battery life.

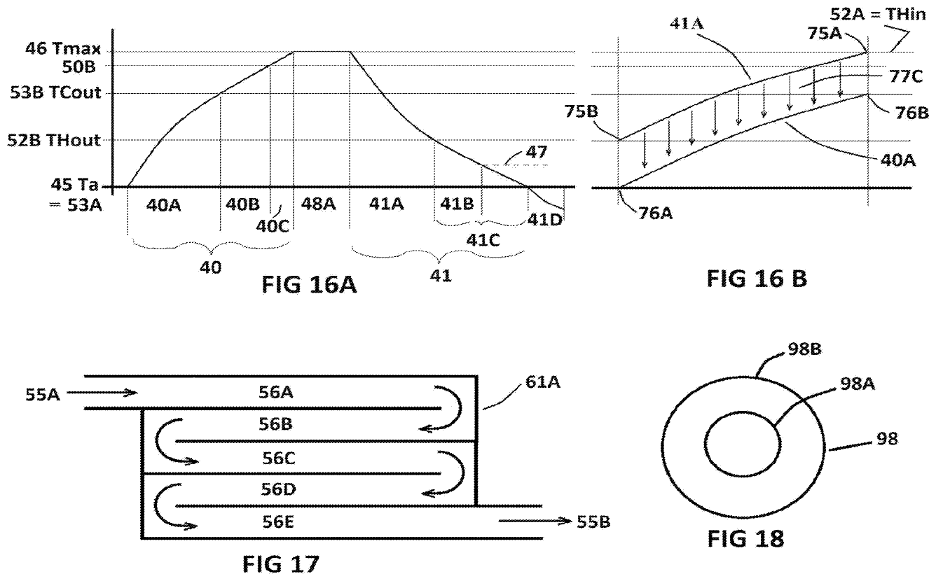

In this embodiment of an air processing deactivating system a heat exchanger 70 is insulated from the ambient with heat exchanger insulator 71 heat exchanger hot air in 75A at temperature hot THin 52A enters the high temperature pipe 73 and exits as heat exchanger hot air out 75B at temperature hot THout 52B. Heat exchanger cold air in 76A at temperature cold TCin 53A enters the low temperature pipe 74 and exits at heat exchanger cold air out 76B at temperature cold TCout 53B. Heat flow out of the high temperature pipe 77A from the hot air stream 75C is transferring heat flow into the low temperature pipe 77B and the cold air stream 76C via the heat transfer fins 72. The temperature profile of this process is depicted in FIG. 16B.

FIG. 15 depicts system components of one embodiment of an air processing deactivating system. Ambient air 19 enters a filter 59. From the filter 59 output the system airflow air 37 is also the cold air stream 76C entering the heat exchanger 70 at temperature cold TCin 53A, and is preheated to temperature cold TCout 53B at the heat exchanger output. From here the system airflow air 37 enters Heater H1 64A which is the first heater in the heater section 60. The system airflow air 37 is heated to heater 2 input temperature 50B. The system airflow air 37 enters heater H2 64B, which is the second heater of the heater section 60, and the system airflow air 37 is heated to the maximum temperature Tmax 46. In this embodiment the system airflow air 37 enters the temperature sustaining heat reservoir reservoir 61. Shown are two representative right and left lengths of reservoir air pipe 67B shown for example only. It is desirable for the reservoir temperature TRout 51D (from FIG. 14) to equal the maximum temperature Tmax 46 from heater section 60 so the temperature can remain constant or higher than it needs to be to ensure even greater pathogen killing potential. From here the system airstream air 37 forms the hot air stream 75C within heat exchanger 70. The hot air stream 75C transfers heat to the cold air stream 76C, thus preheating the cold air stream 76C, and in doing so the hot air stream 75C cools to an output temperature hot THout 52B, whereupon the system airstream air 37 enters the air cooler 22 and exits at temperature cooler out Tco 54A. The system airstream air 37 then enters the pressure out fan 30 which feed the connecting hose 2 and ultimately the positive pressure facemask 3A or positive pressure facemask with attachment elastic adjustable straps 3. The air cooler 22 has both passive and active components, and depending on the temperature cooler out Tco 54A the cooling fan 23 can be turned on and off or modulated via speed control as necessary. The faster the cooling fan 23 spins the greater the temperature reducing capacity of the air cooler 22.

FIG. 14 introduces slightly different nomenclature to describe the four temperatures in the two air streams of an air processing deactivating system. Shown is the input circuit ambient-in air at temperature TCin, or temperature cold in and TCout which is the preheat temperature out. The input circuit airstream absorbs heat from the output circuit which is the hot in feed. The hot feed airstream enters at temperature THin and exits at THout. So THin−Thout=ΔThot will be proportional to the energy or enthalpy lost in the high temperature feed and correspondingly TCout— TCin=ΔTcold will be proportional to the energy or enthalpy increase in the ambient in input circuit. Enthalpy H=U+pV, where U=the thermal energy of the air, p=the pressure, and V=the volume, which in a continuous flow system is volume per unit of time. The internal energy U=mCpT, where m=the mass or mass per unit of time, Cp is the specific heat at constant pressure, and T is the temperature. It is the temperature differential ΔT that is responsible for the −ΔHhot in the hot in circuit and ΔHcold in the cold circuit

In a lossless system—Hhot=Hcold. In a real world system—Hhigh>Hlow, and the greater the insulation value the less heat will be lost from the heat exchanger, and the higher the heat transfer coefficient within the heat exchanger the more efficient the heat exchanger will be. Shown in FIG. 14 is a head to tail configuration which minimizes the temperature differential between the hot and cold circuit, which increases the efficiency but requires a longer length to realize. In this embodiment the highest temperature of the hot feed side transfers heat to the high temperature side of the input cold circuit, and the low or warm temperature side of the hot feed side transfers heat to the low temperature side of the ambient-in feed. This temperature profile is shown in FIG. 16 B. Alternatively, embodiments of the heat exchanger can be configured as a head to head, where the high temperature side of the hot in feed transfers heat to the ambient input cold feed, and thus the temperature differential between the two non-mixing air streams is maximized at the input side. In this embodiment of an air processing deactivating system, the temperature differential decreases as a function of length down the heat exchanger. More heat is transferred at the beginning and less heat at the end, whereas in the head to tail configuration there is a more constant temperature differential throughout the entire heat exchange process. The invention is not limited to either of these embodiments, but the head to tail configuration is shown for example only.

FIG. 16A shows a temperature profile of system airflow air 37 as it passes though the embodiment of of an air processing deactivating system depicted in FIG. 15. The system airflow air 37 exits the air filter 59 at ambient temperature Ta 45 and experiences heating cycle 40, reservoir cycle 48A, and cooling cycle 41. In this embodiment heating cycle 40 encompasses the cold air stream temperature rise in heat exchanger 40A followed by a cold air stream temperature rise due to heater H1 40B which is followed by a cold air stream temperature rise due to heater H2 40C. The temperature at this point is maximum temperature Tmax 46, and this is the temperature entering the temperature sustaining heat reservoir 61 where system airflow air 37 experiences the reservoir cycle 48A. The cooling cycle 41 encompasses a hot air stream temperature fall in heat exchanger 41A (where heat induces a cold air stream temperature rise in heat exchanger 40A) followed by the hot air stream temperature fall to ambient temperature in air cooler 41C. A subset of the hot air stream temperature fall to ambient temperature in air cooler 41C would be the hot air stream temperature fall to temperature above ambient in air cooler 41B if the output temperature of the system airflow air 37 in the air cooler 22 does not cool all the way to ambient temperature Ta 45. In still other embodiments of the invention the system airflow air 37 will be further cooled to supplemental temperature fall below ambient temperature 41D.

FIG. 16B depicts an example of a temperature profile of the cold air stream temperature rise in heat exchanger 40A of an air processing deactivating system and hot air stream temperature fall in heat exchanger 41A within the heat exchanger 70. The hot air stream temperature fall in heat exchanger 41A results from the thermal gradient between the hot air stream 75C and the cold air stream 76C, and this thermal gradient is the driving force for heat flow from hot air stream to cold air stream 77C. The heat exchanger hot air in 75A enters the heat exchanger 70 at maximum temperature Tmax 46 and exits as heat exchanger hot air out 75B at temperature hot THout 52B. The cold air stream temperature rise in heat exchanger 40A results from the thermal gradient between the hot air stream 75C and the cold air stream 76C, and this thermal gradient is the driving force for heat flow from hot air stream to cold air stream 77C. The heat exchanger cold air in 76A enters the heat exchanger 70 at ambient temperature Ta 45 and exits as heat exchanger cold air out 76B at temperature cold TCout 53B. As can be seen schematically represented, heat is transferring continuously down the length of the heat exchanger 70, with the hot air stream 75C dropping continuously in temperature as the cold air stream 76C is continuously increasing in temperature.

FIG. 17 depicts one configuration of a heat reservoir 61A of an air processing deactivating system composed of slats within a rectangular box, thus creating rectangular tubes or channels through which the air passes during its residence time within the heat reservoir 61A. Not shown are heaters and temperature sensors, and this figure is shown for example only. It shows how a rectangular volume can be efficiently utilized as a flow path length increasing reservoir in which the system airflow air 37 enters at the heat reservoir in 55A, travels to the right down the first channel 56A, then to the left down the second channel 56B, then to the right down the third channel 56C, then to the left down the fourth channel 56D, and finally to the right down the fifth channel 56E, and to the heat reservoir out 55B.

FIG. 18 depicts the cross-section of one embodiment of an air processing deactivating system of a tube within a tube heat exchanger 98. Shown is inside tube 98A and outside tube 98B. It can be configured where the inside tube 98A contains the hot airflow feed 98C and the and outside tube 98B contains the cold airflow feed 98D. It can also be configured where the inside tube 98A contains the cold airflow feed 98D and the and outside tube 98B contains the hot airflow feed 98C. The first configuration will be more efficient and produce fewer losses due to a lower thermal gradient with the ambient.

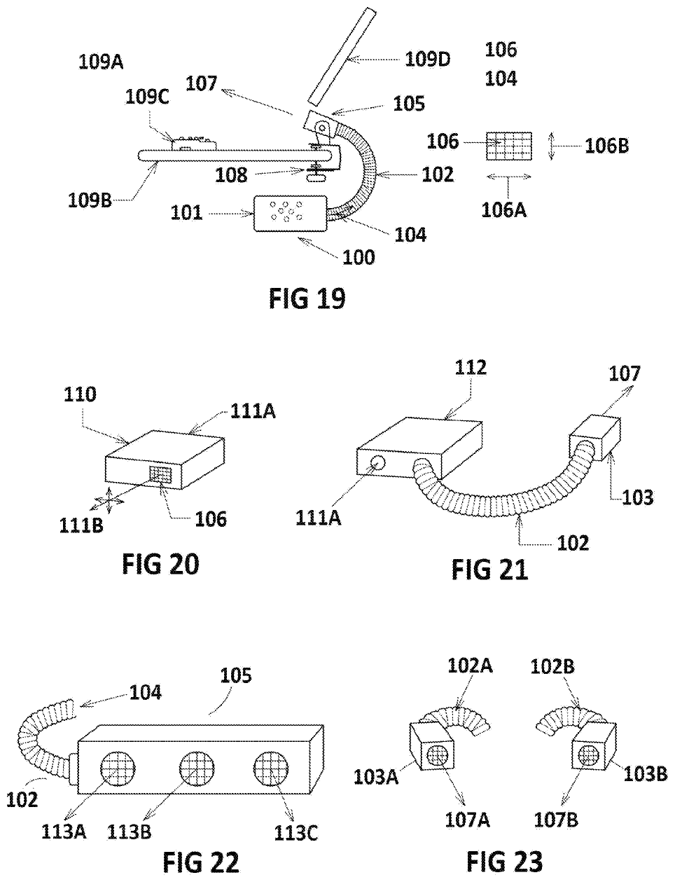

FIG. 19. depicts one embodiment of a non-portable air processing system 100 that can be mounted within a workstation 109A area. Shown is a desk 109B, keyboard 109C, and computer monitor 109D. Ambient air in 101 is drawn into the air processing system 100 from one location and exits through an output hose 102 to a remotely-positionable exit nozzle 103 directing a flow of processed air 104 into the face of an operator. In such a system power consumption is of less concern than in a portable air processing system that must be battery powered. In this embodiment an air processing system 100 takes ambient air from one location, processes it in at least one way as depicted herein with the portable air processing system 1A, and blows the output air into an output hose 102 which directs the air to at least one manifold 105 that can direct air toward the face of an operator. A clamp 108 secures at least one manifold 105 to a desk 109B, shelf, or other protrusion in which the clamp can be secured. The remotely-positionable exit nozzle 103 has adjustable louvres 106 that can be set to direct air to the left or right or up or down. This provides an operator with a processed stream of output air 107 emanating from at least one direction. In this embodiment at least one manifold 105 is a remotely-positionable exit nozzle 103, but at least one manifold 105 can be attached to the output hose 102 as depicted in FIG. 22. Not shown is cooling air for the cooling cycle that can come from a remote location via an attachment hose. Heat can also be expelled in a remote local such as out a window in air-conditioned spaces so an air processing deactivating system will not add heat to the room. These features can be employed in any of the embodiments of either air processing system herein described.

FIG. 20 depicts one embodiment of a non-portable self-contained desktop air processing system 110. This non-portable self-contained desktop air processing system 110 has at least some of the major system components described herein. The non-portable self-contained desktop air processing system 110 takes ambient air in 111A from at least one location and directs processed output air 111B in at least one other direction with adjustable louvres 106.

FIG. 21 depicts one embodiment of a non-portable self-contained desktop air processing system feeding a hose 112, which takes ambient air in 101, processes the air employing at least some of the key heating and cooling steps, then output processed air 104 to an output hose 102 which feeds a remotely-positionable exit nozzle 103. Ambient air in 101 from at least one location, shown here as one location on the back of the non-portable self-contained desktop air processing system feeding a hose 112, feeds processed stream of output air 107 through an output hose 102 to a remotely-positionable exit nozzle 103 containing adjustable louvres 106 capable of adjustably directing the angle of the processed stream of output air 107. The remotely-positionable exit nozzle 103 can also accommodate a clamp 108. And the output hose 102 can feed at least one manifold 105 as depicted in FIG. 22.

FIG. 22 depicts an output hose 102 with flowing processed air 104 as depicted in FIGS. 19 and 21 entering at least one manifold 105 with output direction-directable manifold louvers A 113A, manifold louvers B 113B, and manifold louvers C 113C. The at least one manifold 105 can also incorporate at least one clamp 108. This could have applications in workstations, desks, busses, cars, trains, planes, waiting areas, bars, restaurant tables, bathrooms, casinos, doctor and dentist offices, cashier stations to mention a few.

FIG. 23 depicts first output hose 102A feeding a first remotely-positionable exit nozzle with adjustable louvres 103A producing a first processed stream of output air 107A and a second output hose 102B feeding a second remotely-positionable exit nozzle with adjustable louvres 103B producing a second processed stream of output air 107B. Attachment clamps can be used for each nozzle. This is a useful configuration for workstations where air is directed at an operator from two sources and directions.

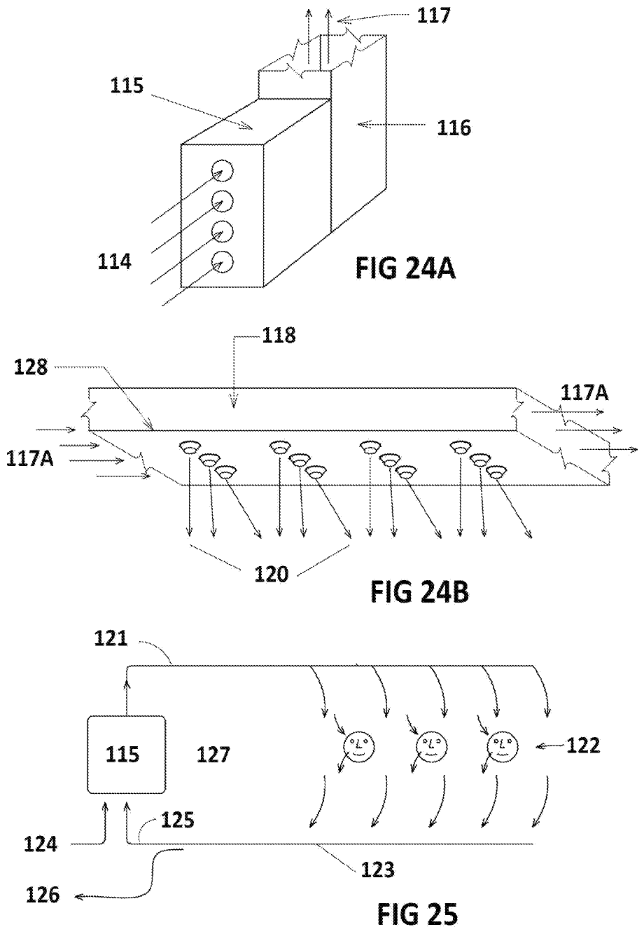

FIGS. 24A and 24B depict some elements in a large installation for processing and distributing large quantities of air to many people such as people in a plane, train, bus, or other controlled or random seating or standing arrangement. Shown is input air 114 entering a central air processing system 115. This central air processing system 115 can contain any of the subcomponents described herein, including but not limited to at least one input mechanical filtration subsystem, at least one UV sterilization subsystem, at least one heat exchanger preheating subsystem, at least one heating subsystem, at least one air reservoir or temperature-controlled air reservoir subsystem, at least one output to at least one heat exchanger subsystem as part of at least one preheat subsystem of an air processing deactivating system, at least one air cooling subsystem of an air processing system, and at least one output air pressure changing and air velocity increasing subsystem that forces air into at least one exit duct 116 for distribution of output processed air 117, at least one air distribution duct 118 containing a multiplicity of exit nozzles or air exit zones 119 through which distributed output air 120 exits into the ambient. In such systems the goal is to create a source of freshly distributed output air 120 that is close to where people breathe, and exhaust air is drawn away from where people breath fresh air as depicted in FIG. 25. At least one air distribution duct 118 is being fed by output processed air in duct 117A and passing further downstream output processed air in duct 117A. In some embodiments the bottom of at least one air distribution duct 118 will have the air output side aligned with the plane of ceiling 128.

FIG. 25 depicts an airflow philosophy in a large space 127 wherein air is processed in a central of an air processing deactivating system 115, exits as output processed airstream 121 which generally blows in one direction, in this case from above, people inhaling and exhaling 122 then breath the output processed air 117, and exhaled air then flows into an exhaust airstream 123 that either exhausts all or a portion of the air to the ambient or draws all the air or a portion thereof back into the air processor for recirculation and reprocessing. Ambient air in 124 is drawn into the central air processing system 115 or mixed with a portion of recirculate air in 125, and any level of mixing can be caused. The exhaust air stream 123 splits into either recirculate air in 125, exhaust air out 126, or any mixture of the two to be mixed with ambient air in 124.

The goal here is to create a flow system of air in a large space 127 that minimizes exhaled breath from being breathed by other people inhaling and exhaling 122. There is no substitute for intelligent positioning based on one's height. Tall people have an advantage being closer to an upward source of downward flowing air, and children have a disadvantage being closer to the floor and thus receiving air from exhaled people above them. However maintaining appropriate distance from people inhaling and exhaling 122 based on height could integrate itself into normal social behavior in the event of pandemics. Air can also emanate from the floor and exhaust from above. In clean rooms where precision assembly occurs, the more sensitive assemblies occur closer to the filtered air entering the room and flowing parallel to the floor and ceiling, and the less sensitive assembly operations occur further downstream where the air grows successively more polluted with particulate material as air gets closer to the room exhaust. For infection control horizontal flow would be less desirable and preferred flow would either be from the ceiling to the floor or the floor to the ceiling.

In one embodiment of an air processing system attachment can comprise a housing with an input orifice, an output orifice, at least one point and method of attachment to a person or animal or method of suspending or standing on a surface. The method of attachment can be loops that a belt can fit through, Velcro that can attach to a belt, hooks, or latches that snap together can be used. The housing can have a shoulder strap or attachable shoulder strap as well. Systems can attach to wheelchairs of the motorized and non-motorized variety. Housings can also attach to bicycles. In other embodiments the housing can simply be placed on a table top surface or on a night table close to a bed. In other embodiments housings can be suspended from the same type hooks as IV bags. The housing can contain at least one battery or power source. For instance, lithium ion batteries are very versatile and have high energy per unit of volume or weight, but lithium polymer batteries are capable of providing very high surge currents, and some embodiments of the invention may employ dual technology batteries so high current peak loads can be drawn from higher surge capacity batteries in a system. Other rechargeable battery technologies can include but are not limited to lead acid, gel cell, nickel metal hydride, nickel cadmium, or other technologies known or unknown. The housing can contain a controller. The housing can contain within the thermal control circuit at least one temperature sensor. In most embodiments there could be several temperature sensors as will be described herein. The invention can contain an input tube connected to the input orifice enabling air to be taken from the ambient. The input tube can also be referred to as a pipe or channel or anything with a closed perimeter containing a cross-section in which system air can flow between any two points within the system. The invention can contain at least one heat source or heating section controlled by the controller and capable of heating the air to a settable or preset maximum or pre-determined temperature of heater output air. The settable temperature can be user-settable on the system itself using a rheostat or settable via WIFI or Bluetooth and controlled from at least one Smart Device. The invention also contains a cooling subsystem producing cooler output air that can cool the heater output air to a comfortable breathing temperature. This temperature may be higher than the ambient temperature, and thus it may not be necessary to cool the heated air to the starting ambient temperature. Other embodiments will enable breathable air to enter the facemask at temperatures lower than the ambient. The invention can have at least one pressure out fan or blower capable of drawing air from the cooler output air and propelling the cooler output air out of the output orifice. The pressure out fan can either be an axial fan or a centrifugal blower to produce higher static pressure rise. The pressure out fan can be at least one pressure increasing fan or blower that can be located anywhere within the airflow system. For instance, there can be a pressure increasing fan or blower located after at least one input filter, and there can be other pressure increasing fans or blowers anywhere between the facemask and any portion of the system at a low enough temperature to enable the fan or blower to operate safely. There is a connecting tube with a first end connecting to the output orifice and a second end connecting to a facemask or other controlled and contained volume within which breathing can occur. This can be a quick connect and disconnect attachment to enable quick and easy component replacement. The quick connect can have a locking feature to prevent an accidental disconnect. The facemask is fitted over a wearer's face to cover the nose and mouth and remain sufficiently sealed around the face contact perimeter so as to enable breathing air supplied with enough positive pressure to prevent accidentally drawing in air from the ambient.

The heating section of an air processing deactivating system can be an insulated heating section to minimize heat transfer from the heating section to the ambient and thus allow more heat to enter the airstream. Further, the heating section of an air processing deactivating system can be isolated and insulated from the cooling subsystem such that there is minimized heat transfer between the two sections, which would be wasteful. In some embodiments of an air processing deactivating system the heaters contained within the heating section are electric powered. In other embodiments it may be desirable for the heating to come from chemical sources such as including but not limited to propane, butane, hydrogen, gasoline, white gas such as the fuel used in Colman lanterns, kerosene, alcohol, or any flammable gas, liquid, or solid fuel. Portable air processing systems can run for periods in excess of eight (8) or more hours using chemical fuel, which frees up the battery for only cooling air. Such systems would work well for the military, first responders, rescue workers, police, firefighters, transit workers, and other people forced to be in the proximity of other people where self-isolation is not possible. Enhanced electric powered air processing systems can also run for longer periods of time for the professional market.

In addition, an intermediate material possessing thermal inertia system can be employed as the material that transfers heated or cooled air to the air to be breathed. This can be useful because it can guarantee that a maximum predetermined temperature cannot be exceeded. This could work with chemical fueled embodiments wherein the chemical heater heats the water to a set point that can be as high as 212 degrees F. or 100 degrees C., and when the water cools below this set point to a lower temperature limit, the chemical heater heats the water back to the upper predetermined limit. The advantage of such embodiments is that the air could never exceed the upper setpoint temperature, and thus the worst-case maximum cooling requirements will always be known. The invention is not limited to water as a working reservoir material, and can be any suitable liquid, solid, liquid and solid, suspension, or at least one gas in whole or in part as a combination of any of the above mentioned. In chemically fueled heaters it is desirable to have thermal overload protection to kick in should temperatures exceed at least one maximum temperature or fall below at least one minimum operating temperature. Redundancy in thermal protection is also anticipated.

In such an embodiment the air processing system would contain a housing with an input orifice, an output orifice, at least one point and method of attachment to a person or animal or method of suspending or standing on a surface. It would also contain at least one battery or power source, a controller, at least one temperature sensor. There would be an input tube connected to the input orifice enabling air to be taken from the ambient. In an embodiment of an air processing deactivating system there would be at least one heat source or heating section controlled by the controller and capable of heating the air to a predetermined maximum temperature of heater output air and a predetermined exposure of time to the predetermined maximum temperature. embodiment of an air processing deactivating system the heat source may incorporate at least one chemical heater employing at least one exothermic chemical reaction to produce heat. This embodiment would also contain a cooling subsystem producing cooler output air that cools the heater output air to a predetermined comfortable breathing temperature. In all embodiments of an air processing system there is at least one pressure fan or blower within the system or attached to the system capable of drawing air from the input orifice to the output orifice. In some embodiments of an air processing system there is a connecting tube with a first end connecting to the output orifice and a second end connecting to a facemask where breathing can occur or attached to a controlled or contained volume or other system. The facemask can fit over a wearer's face to cover the nose and or the mouth and remain sufficiently sealed around the face contact perimeter so as to enable breathing air that is supplied with enough positive pressure to prevent accidentally drawing air from the ambient environment.

Stationary installations can be heated with solar energy of any type that can cause temperature rise. Heat can also be stored in a heat reservoir and transported to provide thermal energy while on the road. Such embodiments would require raising the temperature of a heat storing material to subsequently be carried and drained of thermal energy on demand within a portable system.

In an air processing system a battery can be included within or without such that the battery can be a rechargeable battery and rechargeable from at least one external power source. The battery can be recharged from at least one USB port, though USB ports typically can provide only 1/2 amp of current at 5 volts. Since 12-volt charging is required and a 12-volt battery can be 6 amp hours, USB charging could take over 30 hours, which may not be acceptable, but it is still possible. This would mean charging should most likely be via the use of a dedicate charging system so that a battery could be completely charged within 6 to 8 hours. A typical scenario would be to commute to work using public transportation. At work you would throw the system on the charger, which would fully charge the battery for the homebound commute. In some embodiments the battery can be replaceable as well, and there can be spare batteries for a quick swap or to replace batteries that have exceeded their useful life and no longer hold a charge long enough to be practical. In some embodiments other batteries can be wired in parallel with a separate jack or using the charging jack.

The air processing system can further comprise at least one mechanical filter somewhere before the input orifice, within the body of the system, or after the output orifice. This could in some embodiments be or include a HEPA filter capable of keeping very small particulate material from entering the system. The filter can also incorporate a granulated carbon filter. This mechanical filter can be entirely replaceable. This mechanical filter can have at least one replaceable component such as the filter itself. The mechanical filter can have a quick disconnect feature to allow easy replacement of the entire filter. The mechanical filter can have the ability to be easily cleaned. And the mechanical filter can have the ability to be boiled, which may be a convenient way to clean the system during weekly maintenance or maintenance following a prescribed schedule.

In some embodiments the connecting tube is a tube or hose and the terms are interchangeable. The connecting tube or hose can be a replaceable connecting tube employing a quick connect fitting on the first end that allows quick connection to a mating fitting at the output orifice and a quick connect fitting on the second end that allows quick connection to a mating fitting on the facemask side. There can be a locking feature to prevent accidental disconnect. There are a number of quick connect airflow attachments presently in use, like the fittings used in compressed air systems powering pneumatic tools. There are also quick connect air or gas tube fittings used in medical equipment.

There can be at least one airflow sensor that provides at least one input to the controller.

The cooling subsystem can either be a passive cooling system requiring no external power or a forced-air cooling subsystem possessing at least one fan that is controlled by the controller. The cooling subsystem can be a metal pipe with cooling fins to help draw off heat from the air flowing within. The cooling subsystem can also have flow disrupters to aid in bringing internal streamlines of flowing air to the walls of the pipe to increase the temperature differential and thus speed heat transfer from the air to the ambient.

In the following the term facemask or mask can be a facemask or portion of a facemask that covers the nose and mouth, a face shield, full face covering making good contact with the face or head or a portion thereof, an entire covering for the head, or other volume that isolates a wearer's nose and mouth as a source of inhaling or exhaling.

The facemask can have at least one elastic strap that can fit behind the wearer's head to firmly secure the facemask to the wearer. These straps can be adjustable to obtain the desired holding force of the mask to the wearer's face. The facemask can be a replaceable facemask. The facemask can be a cleanable facemask. The facemask can be a hooded facemask or volume that encloses the entire head. The facemask can be a facemask that can be boiled, as stated above.

In one embodiment, the mask is comprised of a hard outer shell and a soft inner cushion. The hard outer shell allows the attachment of the hose from the portable housing or the output orifice to the mask. The hose attaches and locks to the mask. The inner cushion touches the face and creates a partial seal against the face. The outer harder shell covers most of the inner cushion from viewing. The outer harder shell attaches to the inner cushion securing it in place.

In one embodiment, the mask is comprised of a clear seethrough outer shell and a soft inner cushion. The clear outer shell allows the attachment of the hose from the portable housing or the output orifice to the mask. The hose attaches and locks to the mask. The inner cushion touches the face and creates a partial seal against the face. The outer clear shell attaches to the inner cushion securing it in place.

The facemask can have at least one one-way valve to allow air to flow from the mask (through the outer shell) interior to the ambient during an exhale cycle and to not permit air to flow from the ambient to the facemask interior during an inhale cycle.

In one embodiment of an air processing deactivation system at least one heat source can be a first heat source with a first airflow input of temperature Ta and a first airflow output of temperature Tb. This can be followed by a second heat source with a second airflow input of temperature Tb (the same as the output of the first heater) and a second airflow output of temperature Tc. The first heat source can be the result of a known heater energy dissipation E1 to produce the first temperature rise of Tb−Ta, and this can be used to calculate in real time the amount of energy E2 to be used in the second heat source to produce the target final maximum temperature based on the relationship

E2=k1(Tc−Tb)E1/(Tb−Ta)

where k1=a correction factor to be experimentally determined.

In one embodiment of an air processing system it can be portable for traveling or non-portable for a stationary application in at least one location.

In one embodiment of an air processing system it can further comprise the ability to incorporate a supplemental oxygen supply system.

In one embodiment of an air processing system it can further comprise the ability to incorporate a supplemental humidification subsystem, which can be desirable in dry environments.

In one embodiment of an air processing system it can further comprise the ability to be incorporated within a CPAP system or machine.

In one embodiment of an air processing system it can further comprise at least one of at least one docking station to accommodate connection to at least one Smart Device, at least one of any type USB connector, at least one of at least one other connector enabling connection to at least one Smart Device. Because there will be a fairly large capacity battery in the air processing system it can be used to charge cell phones or other battery powered devices. Not only can this permit charging, but the connector can permit wire communication between at least one Smart Device and the air processing system. This can allow at least one Smart Device to be part of the control system for the air processing system. Communication can be one-directional or bidirectional. This connection can allow the air processing system to become part of a much larger control and data collection and data dissemination system. Records of breathing history can be kept and monitored for changes as well as the integration of the air processing system within a larger harvesting of biometric data.

The air processing system can further comprise at least one one-way valve or drain to enable the removal of accumulated fluid or moisture from at least one portion of the airflow system.

The controller can incorporate adaptive learning based on the breathing habits of the wearer. Adjustments can be made in real-time to heat and cool at a rate that can better track the variable airflow through the system.

The air processing system can further comprise an accumulator or bag in line with the output orifice or anywhere within the output tube as a T intersection such that the accumulator or bag can inflate or deflate as air is breathed by the wearer. The accumulator or bag can be located anywhere downstream of the pressure out fan or blower.

The controller can further comprise WIFI or Bluetooth communication with at least one Smart Device, tablet, laptop, computer, or network to allow the uploading and/or downloading of useful data. This can be one directional or bi-directional communication.

In other embodiments of the air processing system they can further comprise at least one alarm. This alarm can be an audio alarm located on the system itself. The alarm can also be at least one alarm communicated via WIFI or Bluetooth to at least one Smart Device. Or at least one alarm can be multiple alarms simultaneously. At least one alarm can indicate any of the following conditions but is not limited to any of the following conditions stated. An alarm can indicate a disruption of airflow in at least one location within the system.

An alarm can indicate at least one high temperature condition that could create air too hot to breathe. An alarm can indicate at least one low temperature condition that prevents air from achieving the desired target high temperature. An alarm can indicate at least one heater failure. An alarm can indicate at least one fan or blower failure. An alarm can indicate at least one low battery condition. An alarm can indicate at least one battery failure condition. An alarm can indicate at least one improper voltage or current or voltage and current condition. An alarm can indicate at least one component that requires replacing or servicing. An alarm can indicate at least one sensor failure. An alarm can indicate at least one elapsed time that has passed to indicate a requirement to service or clean at least one system component. An alarm can indicate at least one smoke condition. An alarm can indicate at least one fire condition. An alarm can indicate at least one bad air condition. An alarm can indicate at least one low oxygen condition. An alarm can indicate at least one carbon monoxide condition. An alarm can indicate at least one carbon dioxide condition. An alarm can indicate at least one medical alert condition or biometric parameter revealed by the electronic processing of at least one biometric sensor. An alarm can indicate at least one emergency condition. An alarm can indicate at least one operation time remaining before at least one battery discharge condition. An alarm can indicate at least one system malfunction or failure to provide the necessary air at the time it is needed. An alarm can indicate at least one battery fully charged condition. An alarm can indicate at least one failure of at least one battery to accept a charge.

In addition to an alarm the air processing system can further incorporate an automatic airflow shutdown to prevent air that is too hot to breathe from entering the facemask or output tube. This can be either in the form of a temperature reading and the computer shutting off the output flow, or it can be a valve that closes, or it can be a purely mechanical valve that works on a bimetallic metal principal that circumvents the computer altogether in the event of a computer or sensor malfunction.

The air processing deactivation system can further comprise at least one flow disrupter within at least one portion of the heating section and the cooling section of the heat transfer sections of the system. As stated, this would produce turbulence within the system and mix the air within the air transport pipe.

In an embodiment of the air processing system the user can adjust at least one temperature or the user can also adjust the rate of the circulation or feedback path of air from at least one location within the system to at least one other location within the system to mix at least one stream of air with at least one other stream of air. In an embodiment of the air processing system the user can also adjust at least one fan speed. In an embodiment of the air processing system the user can also adjust the air feedback to incoming air ratio to increase or decrease the output airflow into the mask of the user.

The air processing deactivation system can further comprise a heat exchanger. In its most basic form a heat exchanger exchanges heat between a high temperature flow of a fluid and a low temperature flow of a fluid. Heat flows from the high temperature source to the low temperature receiving fluid. The high temperature side consists of an airflow heat loss circuit possessing a loss airflow input at a loss input temperature and a loss airflow output at a loss output temperature. The low temperature side consists of a heat gain circuit possessing a gain airflow input at a gain input temperature and a gain airflow output at a gain output temperature. The heat gain circuit is capable of preheating the airflow at at least one point within the system flow that is before the entrance to at least one heat source. The heat loss circuit is capable of cooling the airflow after and at the output of at least one heat source or at at least one point within the system that is located after at least one heat source. The heat loss circuit transfers heat to the heat gain circuit to preheat the airflow while lowering the temperature of loss output temperature. Lowering the temperature of loss output temperature decreases the cooling requirement of the air cooling portion of the system, so the more efficient the heat exchanger is, the less cooling is required and the less heating is required of the input air because the input temperature to the heating portion of the system will be higher.

The air processing deactivation system can further comprise at least one air reservoir capable of sustaining at least one temperature produced by at least one heat source or heating section. The air reservoir can be composed of at least one length of pipe or channel possessing at least one cross-sectional shape capable of increasing the distance air must flow, and thus increasing the residence time the air spends at at least one temperature. The reservoir can further comprise at least one temperature sensor. The reservoir can further comprise at least one reservoir heater to heat air in at least one region as air passes through said reservoir. The reservoir can further comprise at least one insulating material insulating the reservoir from at least one other component within the air processing system.

In another embodiment the air processing system a housing with an input orifice, an output orifice is included, and at least one point and method of attachment to a person or animal or method of suspending or standing on a surface. The embodiment the air processing system also has at least one battery or power source, and a controller for controlling system elements. In one embodiment the air processing system there is an input filter to filter ambient air prior to entering the input orifice. This is followed by an input tube connected to the input orifice that enables air to be taken from the ambient after passing through the input filter. From there the air passes through at least one heat source or insulated heating section controlled by the controller, and this heating section is capable of heating the air to a settable or preset maximum temperature of heater output air. The settable temperature can be set by the user via a rheostat or this temperature can be set via a Smart Device via wired connection or via WIFI or Bluetooth. This heater output air then feeds a cooling subsystem that can cool the heater output air to a comfortable breathing temperature. This is followed by at least one pressure out fan or blower capable of drawing air from the cooler output air and propelling the cooler output air out of the output orifice. A connecting tube with a first end connects to the output orifice and a second end connects to a facemask or other controlled and contained volume within which breathing can occur. The facemask can fit over a wearer's face to cover the nose and mouth and remain sufficiently sealed around the face contact perimeter so as to enable breathing air supplied with enough positive pressure to prevent accidentally drawing air from the ambient.

In another embodiment the air processing system comprises a housing with an input orifice, an output orifice, at least one point and method of attachment to a person or animal or method of suspending or standing on a surface. It can have at least one battery or power source. It can have a controller. It can have at least one temperature sensor.

In another embodiment the air processing system can have one of the following: an input tube connected to said input orifice enabling air to be taken from the ambient or an input filter that filters air from the ambient and feeds the input orifice which feeds the input tube, or the input tube can feed at least one heat source or heating section controlled by the controller and capable of heating the air to a settable or preset maximum temperature of heater output air, or a heat exchanger with an input to a preheat circuit fed by the input tube, and an output from the preheat circuit that feeds at least one heat source or heating section controlled by the controller and capable of heating the air to a settable or preset maximum temperature of heater output air.

In another embodiment the air processing deactivation system can have one of the following: a cooling subsystem producing cooler output air that cools the heater output air to a comfortable breathing temperature, or a heat exchanger with an input to a heat loss circuit taking air from the heater output air and an output from the heat loss circuit that feeds to the cooling subsystem producing cooler output air that cools the air from the output from the heat loss circuit to a comfortable breathing temperature, or an air reservoir capable of sustaining at least one temperature produced by at least one heat source or heating section, and the air reservoir is composed of at least one length of pipe or channel possessing at least one cross-sectional shape capable of increasing the distance air must flow before feeding a cooling subsystem producing cooler output air that cools the heater output air to a comfortable breathing temperature, or an air reservoir capable of sustaining at least one temperature produced by at least one heat source or heating section, the air reservoir being composed of at least one length of pipe or channel possessing at least one cross-sectional shape capable of increasing the distance air must flow feeding a heat exchanger with an input to a heat loss circuit and an output from the heat loss circuit that feeds to the cooling subsystem producing cooler output air that cools the air from the output from the heat loss circuit to a comfortable breathing temperature. It can have at least one pressure out fan or blower capable of drawing air from the cooler output air and propelling the cooler output air out of the output orifice. It can have a connecting tube with a first end connecting to the output orifice and a second end connecting to a facemask or other controlled and contained volume within which breathing can occur. The facemask can fit over a wearer's face to cover the nose and mouth and remain sufficiently sealed around the face contact perimeter so as to enable breathing air supplied with enough positive pressure to prevent accidentally drawing air from the ambient environment.

In other embodiments the air processing system can further comprise at least one regulator or pressure regulator interposed between said facemask and said at least one pressure out fan or blower. The regulator is not limited to this position however and in some embodiments a regulator can be located anywhere within the airflow line between the input where air from the ambient is taken and the facemask. This regulator can act to allow air to flow only during an inhale flow cycle, and when air does flow it flows with positive pressure at the wearer's nose and mouth. This can save power and not waste as much processed air during the exhale cycle. This regulator can function similarly to a mouthpiece SCUBA low pressure regulator. It can be a pressure reducing regulator. It can be a back pressure regulator. It can employ inhale or exhale or inhale and exhale sensing. In some embodiments the system and controller can learn and anticipate the breathing pattern of the wearer, and this can help ensure that there is more air than is necessary as the air demand increases and decreases.

In another embodiment the air processing deactivation system can be an air processing system comprising a housing with an input orifice, an output orifice, at least one point and method of attachment to a surface or the ability to be placed on a surface. It can have the ability to have external power applied. It can have a controller. It can have at least one temperature sensor. It can have an input tube connected to the input orifice enabling air to be taken from the ambient. It can have at least one heat source or heating section controlled by the controller and capable of heating the air to a settable or preset predetermined maximum temperature of heater output air. It can have a cooling subsystem producing cooler output air that cools the heater output air or output air from a reservoir or output air from a heat exchanger to a comfortable breathing temperature. It can have at least one pressure out fan or blower capable of drawing air from the cooler output air and propelling the cooler output air out of the output orifice. It can have a connecting tube or hose with a first end connecting to the output orifice and at least one second end connecting to at least one of: a manifold, a manifold with at least one exit nozzle with air direction louvres, at least one exit nozzle, at least one exit nozzle with air, or direction louvres.

In another embodiment the air processing deactivation system can have at least one of: a heat exchanger with an input to a preheat circuit fed by the input tube or other subsystem, an output from the preheat circuit that feeds at least one heat source or heating section, an input to a heat loss circuit fed by at least one heat source or heating section or a reservoir output, an output from the heat loss circuit that feeds the cooling subsystem.

In another embodiment the air processing system can have a UV sterilizer subsystem fed by the input tube as an input and produces an output that feeds the input to a preheat circuit of the heat exchanger or feeds at least one heat source or heating section.

In another embodiment the air processing deactivation system can have at least one filter through which ambient air must pass before entering the input tube.

In another embodiment the air processing deactivation system can have a reservoir taking as an input the output of at least one heat source or heating section and outputs air to the cooling subsystem or the input to a heat loss circuit of the heat exchanger.

In another embodiment the air processing deactivation system can comprise an input tube through which air from the ambient is drawn, a heating portion within which the air is heated from the ambient temperature to a maximum temperature that is user-settable or can be preset, a cooling portion within which the air is cooled back to or near the ambient temperature, an exit nozzle through which the air exits, a connecting tube through which the processed air passes on its way to a facemask that fits around a wearer in which to breath the processed air from a predominantly positive pressure supply of processed air.

During normal breathing a person can use 7 or 8 liters per minute of air. The specific heat of air is about 1005 joules/Kg of air (0.24 BTU/pound mass in the English system), and the density of air is about 1.225 Kg/meter\hat{ }3. Thus, the energy required to raise a cubic meter of air 1 degree Centigrade is 1249.5 joules/meter\hat{ }3. At 8 liters of air per minute, a cubic meter of air will permit 125 minutes of breathing. As a benchmark, the energy necessary to raise a cubic meter of air 100 degrees Centigrade is about 124,950 joules without loss, or 999.6 joules per minute, or 16.66 joules per second, which is 16.66 watts average continuous. This is very easily achievable with portable batteries. Without the power required by fans and a processor and support electronics, a 12 volt battery would require an average of 1.388 amps just to heat the air. But breathing is not a continuous operation and occurs in pulses. One can spend half the time inhaling and half the time exhaling. In a minute one can take 10 to 12 breaths. At a 10 breath per minute breathing rate one can breathe 800 milliliters (48.82 cubic inches) of air per breath, and this can produce a velocity pulse of air in addition to the velocity profile throughout the cross-section of a tube. An average breath increases to an approximate maximum or peak inspiration flow rate which is sustained for a short period of time, then the velocity falls to zero prior to exhaling, producing a trapezoidal air velocity profile over the time of a single breath. If for the sake of argument, inhaling and exhaling are equal in length, 30 seconds of one minute's worth of breathing is inhaling and 30 seconds of one minute is spent exhaling. Assuming a constant speed of breathing, the peak power requirement during inhalation would be greater than 33.32 watts, or 2.777 amps at 12 volts. With the other system fans, losses, and electronics overhead the system could average 2.5 to 3 amps continuous, so a 12-volt battery would require a 2.5 to 3 amp hour capacity to run a portable system for 1 hour before battery charging is required. A 6-amp hour battery could provide over 2 hours of operation. If the maximum temperature is lowered the operating time will increase. And depending on the leakage tolerated the air supply will be greater but the operating time will be lower. For a one-hour commute on a train, subway, or bus where breathing demand is not high, 1.5 hours of operation could be expected from a 12-volt 6 amp hour battery. During walking, running, or in a state of panic the air demand will rise precipitously so the design should be capable of meeting a worst-case heating demand, which will shorten the total operating time.

In another embodiment the air processing system can have a heat exchanger. This can increase the run time by 50% or more because the much of the energy lost to cooling can be gained in preheating the incoming air. A heat exchanger will increase the complexity of the system but will also increase the run time and is more energy efficient.

Although preferred embodiments of the present invention have been described herein it will be understood by those skilled in the art that the present invention should not be limited to the described preferred embodiments. Rather, various changes and modifications can be made within the spirit and scope of the present invention.

In another embodiment of an air processing system comprises a housing with an input orifice and an output orifice, wherein the input orifice is configured to receive air from ambient or other component. The input orifice enables air to be taken from the ambient, but air can come from other sources as well. The air processing system can be in series with other external components or subsystems. The input orifice can receive input air from a filter or an oxygenator or other air moving or air processing system. An attachment can be configured to attach to at least one person or an animal. There is at least one battery. There is a controller. The controller can read sensor information and calculate control strategies, generate outputs to turn heaters on and off, fans and blower on and off, power supplies on and off, loads on and off, or supplemental electronics on and off, or control PWM signals to produce variable control to any of the above.

In another embodiment of an air processing deactivation system there is at least one temperature sensor. At least one heat source is controllable by the controller and configured to heat air to a predetermined temperature. The predetermined temperature can be stored in memory or can be settable depending upon the environment or situation. There is a cooling subsystem that cools said heater output air. The cooled temperature can be ambient temperature, a temperature above ambient, or in other embodiments temperatures below ambient. At least one a fan or a blower is configured to draw air from the input orifice to the output orifice. The placement will generally be either before or after the heating section. Some embodiments may require multiple fans or blowers to overcome system fluid resistance and to keep the airflow at the necessary volumetric capacity. A connecting structure has a first end connecting to the output orifice and a second end connecting to a facemask or other volume or subsystem.

In another embodiment of an air processing deactivation system the predetermined temperature generated by the heating source is capable of deactivating at least one Corona virus.

In another embodiment of an air processing deactivation system the predetermined temperature generated by the heating source is capable of deactivating at least one of a virus, an allergen, a pathogen, a bacterium, a protist, an organism, a living cell, or a pollen.

In an embodiment of an air processing system the battery is at least one of: a rechargeable from at least one external power source, a rechargeable from at least one USB port, a replaceable, or has the capability to be connected in parallel with at least one other battery.

In another embodiment of an air processing system it further comprises at least one mechanical filter, and this mechanical filter is configured to be at least one of: replaceable, incorporate a HEPA filter, incorporate a granulated carbon filter, include at least one replaceable component, a quick disconnect feature allowing easy replacement, or have the ability to be easily cleaned.

In another embodiment of an air processing system the connecting structure comprises a replaceable connecting tube employing a quick connect fitting that locks in place on both ends.

In another embodiment of an air processing system can further comprise at least one airflow sensor that provides at least one input to the controller.

In another embodiment of an air processing deactivation system the cooling subsystem can comprise a passive cooling system requiring no electrical power or a forced-air cooling subsystem including at least one fan controllable by said controller.

The cooling subsystem is capable of cooling air to a temperature that is comfortable to breathe.

In another embodiment of an air processing system the facemask is at least one of a replaceable facemask, a cleanable facemask, a hooded facemask or volume that encloses the entire head, a facemask that is cleanable or is dishwasher safe.

The facemask can further comprise at least one one-way valve, at least one filter, or both. In some embodiments air should only flow out of the facemask but not have ambient air flow back into the facemask. In some embodiments it is necessary to pass output air and exhaled air through a filter to catch aerosols emitted from the nose and mouth. And both features can be incorporated in series or in parallel.

In another embodiment of an air processing system the controller can incorporate adaptive learning or artificial intelligence techniques based on the breathing habits and instantaneous demands of the wearer and can make adjustments in real-time to heat and cool at a rate that can better track the variable airflow through the system. This can be taken a step further to supply air leading breathing demand.

In another embodiment of an air processing system there can be an instant airflow shut-down button or electrical signal to initiate airflow shut-off to the facemask in less than ½ second.

In another embodiment of an air processing system the controller can further comprise WIFI or Bluetooth or both forms of communication with at least one Smart Device or other device capable of sending, receiving, or sending and receiving wireless signals.

In another embodiment of an air processing system the facemask can further comprise a microphone to enable the wearer's speech to be heard from an external sound system. This would take audio signals from the microphone and send them wirelessly to external Bluetooth speakers in some embodiments.

The wearer's speech can occur in whole or in part after airflow shut-off to the facemask. The airflow can be manually arrested or a speech signal from the microphone or amplifying electronics can trigger airflow shut-off. When sound stops for a period of time, airflow can automatically resume so the wearer can inhale.

Another embodiment of an air processing deactivation system can further comprise at least one alarm based on at least one of the following: a disruption of airflow, a high or low temperature condition, a heater failure, a blower failure, a filter failure, a bad air condition, a medical alert or abnormal biometric parameter.

Another embodiment of an air processing system can further comprise at least one alarm based on at least one of the following: a disruption of airflow, a high or low temperature condition, a blower failure, a filter failure, a bad air condition, a medical alert or abnormal biometric parameter.

In another embodiment of an air processing system the user can adjust an output temperature or the rate of air flowing into the facemask or both.

In another embodiment of an air processing system there can be at least one input filter, the output of which feeds the input orifice.

In another embodiment of an air processing filtering system there must be at least two separate input filters to filter the ambient air before the input orifice.

In another embodiment of an air processing system the facemask can be a two-piece facemask comprising a soft cushion that contacts the face and an outer shell connecting to the cushion that contains a connector enabling attachment a connecting tube. The outer shell can be soft or hard.

In another embodiment of an air processing filtering system there can be a user selectable turbo mode to cause the airflow to increase to a maximum flow rate to the facemask. Should the user want to be hit with a blast of high volume air this will be a useful feature.

The facemask can further comprise a facemask door or shutter that opens and closes in the outer shell of the facemask. This allows liquid or food or material to gain access to the wearer and to allow speech to occur without anything covering the mouth. This can allow smoking or other movement of materials between the wearer's mouth and the ambient. This door or shutter can be initiated mechanically or electrically. For instance, as a hand holding a fork approaches the facemask, a proximity sensor or detection system can sense the approach and open the door to allow food to enter the mouth, and when the hand and utensil moves away from the face and mask the door will automatically close. This sensing function can be performed optically, with IR, acoustically, visually, or manually initiated to name a few.

In another embodiment of an air processing filtering system the facemask fitting over a wearer's face can cover the nose and mouth and remain sufficiently sealed around the face contact perimeter so as to enable breathing air supplied with enough positive pressure to prevent accidentally drawing air from the ambient.

In another embodiment of an air processing filtering system there can be at least one regulator or pressure regulator interposed between the facemask and at least one pressure out fan or blower or anywhere between the facemask and the cooling section.

In an embodiment for installation to process air for a building, a train, a vehicle, or a plane, the air processing system comprises a housing with an input orifice and an output orifice, a support frame or mounting system for installation in at least one of a building, a train, a vehicle, a plane, the ability to have external power applied, a controller, at least one temperature sensor, at least one filter allowing air to pass from the ambient to the input orifice, at least one heating subsystem controlled by the controller capable of heating the air to a predetermined temperature of heating subsystem output air, a cooling subsystem producing cooler output air that cools said heating subsystem output air or output air from a reservoir or output air from a heat exchanger to a comfortable breathing temperature, at least one pressure out fan or blower capable of drawing air from at least one of the cooling subsystem output air, output air from the input orifice and propelling the cooler output air out of output orifice. There is also at least one heat exchanger with an output circuit fed by air from the heater subsystem and an input circuit fed by input air prior to heating. There can be a heat reservoir in some embodiments.

In another embodiment of an air processing system there is at least one output manifold to distribute air to a building, a train, a vehicle, a plane or other place where there are many people.

1. An air processing system comprising: a portable housing with an input orifice and an output orifice, wherein the input orifice is configured to receive air from ambient or other component; an attachment configured to attach to at least one person or an animal; at least one battery; a controller; at least one temperature sensor; said input orifice enabling air to be taken from the ambient; at least one heat source controllable by said controller and configured to heat said air to a predetermined temperature; a cooling subsystem that cools said heater output air; at least one of a fan or a blower configured to draw air from the input orifice to the output orifice; a connecting tube having a first end connecting to said output orifice and a second end connecting to a facemask or other volume or subsystem.

2. The air processing system of claim 1 wherein said predetermined temperature generated by the heating source is capable of deactivating at least one Corona virus.

3. The air processing system of claim 1 wherein said predetermined temperature generated by the heating source is capable of deactivating at least one of a virus, an allergen, a pathogen, a bacterium, a protist, an organism, a living cell, or a pollen.

4. The air processing system of claim 1 wherein said battery is at least one of: rechargeable from at least one external power source; rechargeable from at least one USB port; replaceable; has the capability to be connected in parallel with at least one other battery.

5. The air processing system of claim 1 further comprising at least one mechanical filter, wherein said mechanical filter is configured to be at least one of: be replaceable; incorporate a HEPA filter; incorporate a granulated carbon filter; include at least one replaceable component; include a quick disconnect feature allowing easy replacement.

6. The air processing system of claim 1 wherein said connecting tube comprises a replaceable connecting tube employing a quick connect fitting that locks in place on both ends.

7. The air processing system of claim 1 further comprising at least one airflow sensor that provides at least one input to said controller.

8. The air processing system of claim 1 wherein said cooling subsystem comprises a passive cooling system requiring no electrical power or an electrically-powered forced-air cooling subsystem including at least one fan controllable by said controller.

9. The air processing system of claim 8FF wherein said cooling subsystem is capable of cooling said air to a user settable temperature.

10. The air processing system of claim 1 wherein said facemask is at least one of a replaceable facemask, a cleanable facemask, a hooded facemask or volume that encloses the entire head, a facemask that is cleanable or is dishwasher safe.

11. The air processing system of claim 1 wherein said facemask further comprises at least one one-way valve, at least one filter, or both.

12. The air processing system of claim 1 wherein said at least one heat source is a first heat source with a first airflow input of temperature Ta and a first airflow output of temperature Tb followed by a second heat source with a second airflow input of temperature Tb and a second airflow output of temperature Tc, said first heat source being the result of a known heater energy dissipation E1 to produce the first temperature rise of Tb−Ta, and this is used to calculate in real time the amount of energy E2 to be used in said second heat source to produce the target final temperature based on the relationship: E2=k1(Tc−Tb)E1/(Tb−Ta), where k1=a system correction factor to be experimentally determined.

13. The air processing system of claim 1 further comprising at least one of: the ability to incorporate supplemental oxygen supply system, the ability to incorporate a supplemental humidification subsystem, the ability to be incorporated within a CPAP system, the ability to recirculate the air back through the device allowing multiple cycles of heating and cooling, at least one of at least one docking station to accommodate connection to at least one smart device, at least one of any type USB connector, at least one of at least one other connector enabling connection to at least one smart device, the above to permit charging or communication or charging and communication; at least one one-way valve or drain to enable the removal of accumulated fluid or moisture from at least one portion of the airflow system.

14. The air processing system of claim 1 wherein said controller incorporates adaptive learning or artificial intelligence techniques based on the breathing habits and instantaneous demands of the wearer and can make adjustments in real-time to heat and cool at a rate that can better track the variable airflow through the system.

15. The air processing system of claim 1 further comprising an accumulator or bag in line with at least one of: the output orifice; anywhere within said output tube as a T-intersection such that said accumulator or bag can inflate or deflate as air is breathed by the wearer; anywhere downstream of said pressure out fan.

16. The air processing system of claim 1 wherein said controller further comprises WIFI or Bluetooth communications with at least one smart device or other device capable of sending, receiving, or sending and receiving wireless signals.

17. The air processing system of claim 1 wherein said facemask further comprises a microphone to enable the wearer's speech to be heard from an external sound system.

18. The air processing system of claim 17 wherein said wearer's speech occurs in whole or in part after airflow shut-off to said facemask.

19. The air processing system of claim 1 further comprising at least one alarm based on at least one of the following: a disruption of airflow, a high or low temperature condition, a heater failure, a filter failure, a bad air condition, a medical alert or abnormal biometric parameter.

20. The air processing system of claim 1 further comprising at least one flow disrupter within at least one portion of the heating section and the cooling section of the heat transfer sections of the system.

21. The air processing system of claim 1 wherein the user can adjust at least one of the following: an output temperature, the rate of air flowing into the facemask.

22. The air processing system of claim 1 further comprising at least one input filter, the output of which feeds said input orifice.

23. The air processing system of claim 1 further comprising at least two separate input filters to filter the ambient air before the said input orifice.

24. The air processing system of claim 1 further comprising a two-piece facemask comprising a soft cushion that contacts the face and an outer shell connecting to the cushion that contains a connector enabling attachment a connecting tube.

25. The air processing system of claim 1 further comprising a user selectable turbo mode to cause the airflow to increase to a maximum flow rate to the facemask.

26. The air processing system of claim 1 further comprising an instant airflow shut-down button or electrical signal to initiate airflow shut-off to the facemask in less than ½ second.

27. The air processing system of claim 1 further comprising a facemask door or shutter that opens and closes in the outer shell of the facemask, said door or shutter to be initiated mechanically or electrically.

28. The air processing system of claim 1 wherein said facemask fitting over a wearer's face to cover the nose and mouth and remain sufficiently sealed around the face contact perimeter so as to enable breathing air supplied with enough positive pressure to prevent accidentally drawing air from the ambient.

29. The air processing system of claim 1 further comprising a heat exchanger with an airflow heat loss circuit possessing a loss airflow input at a loss input temperature and a loss airflow output at a loss output temperature and a heat gain circuit possessing a gain airflow input at a gain input temperature and a gain airflow output at a gain output temperature, said heat gain circuit capable of preheating said airflow at at least one point within the system flow that is before the entrance to said at least one heat source, said heat loss circuit capable of cooling said airflow after and at the output of said at least one heat source or at at least one point within the system that is located after said at least one heat source, said heat loss circuit transferring heat to said heat gain circuit to preheat said airflow while lowering the temperature of said loss output temperature.

30. The air processing system of claim 1 further comprising at least one air reservoir.

31. The air processing system of claim 1 further comprising at least one regulator or pressure regulator interposed between said facemask and said at least one pressure out fan or blower.

32. An air processing system comprising: a housing with an input orifice, an output orifice, at least one point and method of attachment to a surface or the ability to be placed on a surface; the ability to have external power applied; a controller; at least one temperature sensor; an input tube connected to said input orifice enabling air to be taken from the ambient; at least one heating section controlled by said controller capable of heating said air to a predetermined maximum temperature of heater output air; a cooling subsystem that cools said heater output air; at least one pressure out fan or blower capable of drawing air from at least one of said cooling subsystem output air, air to said input tube, output air from said input tube and propelling said cooling subsystem output air out of said output orifice; a connecting tube or hose with a first end connecting to said output orifice and at least one second end connecting to at least one of: a manifold, a manifold with at least one exit nozzle with air direction louvres, at least one exit nozzle, at least one exit nozzle with air direction louvres; and at least one of: a heat exchanger with: an input to a preheat circuit fed by said input tube or other subsystem, an output from said preheat circuit that feeds said at least one heating section, an input to a heat loss circuit fed by said at least one heating section, an output from said heat loss circuit that feeds said cooling subsystem; at least one filter through which ambient air must pass before entering said input tube; a reservoir taking as an input the output of said at least one heat source or heating section and outputs air to said cooling subsystem or said input to a heat loss circuit of said heat exchanger.

33. The air processing system of claim 32 further comprising a UV sterilizer subsystem fed by said input tube as an input and produces an output that feeds said input to a preheat circuit of said heat exchanger or feeds said at least one heat source or heating section

34. An air processing system comprising: a housing with an input orifice and an output orifice; a support frame or mounting system for installation in at least one of a building, a train, a vehicle, a plane; the ability to have external power applied; a controller; at least one temperature sensor; at least one filter allowing air to pass from the ambient to the input orifice; at least one heating subsystem controlled by said controller capable of heating said air to a predetermined temperature of said heating subsystem output air; a cooling subsystem producing cooler output air that cools said heating subsystem output air or output air from a reservoir or output air from a heat exchanger to a comfortable breathing temperature; at least one pressure out fan or blower capable of drawing air from at least one of said cooling subsystem output air, output air from said input orifice and propelling said cooler output air out of said output orifice; a heat exchanger with an output circuit fed by air from said heater subsystem and an input circuit fed by input air prior to heating; a heat reservoir; at least one output manifold to distribute air to said building, a train, a vehicle, a plane.

35. An air processing system comprising: a portable housing with an input orifice, an output orifice; at least one point and method of attachment to a person or animal; at least one battery; a controller; an input orifice enabling air to be taken from the ambient; a user control dial to set the airflow rate to the facemask; at least one fan or blower capable of drawing air through the system from the input orifice to the output orifice; a connecting tube with a first end connecting to said output orifice and a second end connecting to a facemask or other volume; at least two separate input filters in parallel to filter the ambient air before or after the said input orifice.

36. The air processing system of claim 35 further comprising a two-piece facemask comprising a soft cushion that contacts the face and an outer shell connecting to the cushion that contains a connector enabling attachment a connecting tube.

37. The air processing system of claim 35 including a turbo mode button to cause the airflow to the facemask to increase to the maximum deliverable airflow possible.

38. The air processing system of claim 35 further comprising an instant airflow shut-down button or electrical signal to initiate airflow shut-off to the facemask in less than ½ second.

39. The air processing system of claim 35 wherein said facemask further comprises a microphone to enable the wearer's speech to be heard from an external sound system.

40. The air processing system of claim 39 wherein said wearer's speech occurs in whole or in part after airflow shut-off to said facemask.

41. The air processing system of claim 35 further comprising a facemask door or shutter that opens and closes in the outer shell of the facemask, said door or shutter to be initiated mechanically or electrically.

42. The air processing system of claim 35 including a facemask fitting over a wearer's face to cover the nose and mouth and remain sufficiently sealed around the face contact perimeter so as to enable breathing air supplied with enough positive pressure to prevent accidentally drawing air from the ambient.

43. The air processing system of claim 35 wherein said facemask is at least one of a replaceable facemask, a cleanable facemask, a hooded facemask or volume that encloses the entire head, a facemask that is cleanable or is dishwasher safe.

44. The air processing system of claim 35 wherein said facemask further comprises at least one one-way valve, at least one filter, or both.
